(19) United States
(12) Patent Application Publication
(10) Pub. No.: US 2026/0257979 A1
(43) Pub. Date: Sep. 3, 2026

(54) CRYSTAL OF TRISPHENOL COMPOUND AND METHOD FOR PRODUCING THE SAME

(71) Applicant: HONSHU CHEMICAL INDUSTRY CO., LTD., Chuo-ku, Tokyo (JP)

(72) Inventors: Ryo KIMURA, Wakayama-shi (JP); Yusuke IWAIDA, Wakayama-shi (JP)

(21) Appl. No.: 18/881,678

(22) PCT Filed: Sep. 13, 2023
(86) PCT No.: PCT/JP2023/033291

§ 371 Date: Jan. 6, 2025

(30) Foreign Application Priority Data
Sep. 16, 2022 (JP) 2022-147796

(51) Int. Cl. C07C 39/16

(52) U.S. Cl. CPC C07C 39/16; C07B 2200/13

(57) ABSTRACT

An object is to provide means for solving problems with solids and crystallization methods known in the art related to a trisphenol compound represented by chemical formula (A). As a solution, a crystal of the trisphenol compound represented by chemical formula (A) and a method for producing the crystal are provided:

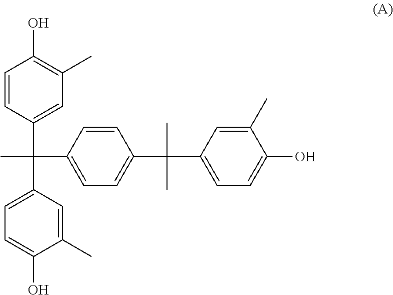 [SMILES: Cc1cc(C(C)(C)c2ccc(C(C)(c3ccc(O)c(C)c3)c3ccc(O)c(C)c3)cc2)ccc1O]

### TECHNICAL FIELD

The present invention relates to a crystal of a trisphenol compound and a method for producing the crystal.

### BACKGROUND ART

Trisphenol compounds are used in, for example, electronic material applications, specifically, as raw materials for photosensitive materials in resist materials, additives intended to help promote or inhibit dissolution in developers, and the like. Under such circumstances, polynuclear phenols are required to be highly pure and not to contain much solvent. When a large amount of solvent is contained, an operation such as solvent displacement is necessary in order to remove the solvent contained, which is disadvantageous from the viewpoint of industrial production.

As one of the trisphenol compounds, a trisphenol compound represented by chemical formula (A) (4,4′-(1-(4-(2-(4-hydroxy-3-methylphenyl)propan-2-yl)phenyl)ethane-1,1-diyl)bis(2-methylphenol), hereinafter also referred to as compound A) is conventionally known (PTLs 1 to 5).

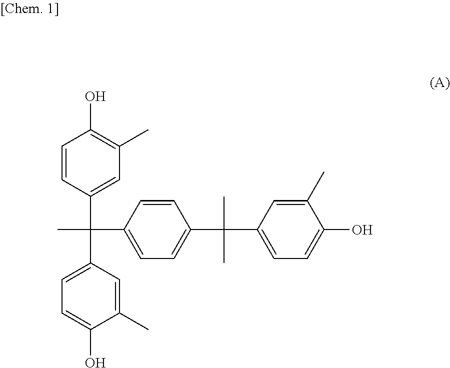 [SMILES: Cc1cc(C(C)(C)c2ccc(C(C)(c3ccc(O)c(C)c3)c3ccc(O)c(C)c3)cc2)ccc1O]

### CITATION LIST

### Patent Literature

PTL 1: Korean Patent Application No. 10-2017-0006311
PTL 2: U.S. Pat. No. 4,954,603
PTL 3: Japanese Unexamined Patent Application Publication No. 62-084035
PTL 4: Japanese Unexamined Patent Application Publication No. 04-251849
PTL 5: Japanese Unexamined Patent Application Publication No. 10-97075

### SUMMARY OF INVENTION

### Technical Problem

In PTLs 1 to 5, Synthesis Examples of compound A are described, and two kinds of crystallization methods are described. Specifically, (1) a crystallization method using a mixed solvent of toluene and MIBK (PTL 1) and (2) a crystallization method using decane as a solvent (PTLs 2 to 5) are described, and crystals of compound A obtained by these crystallization methods are described as having melting points of 87° C. to 91° C.

This time, regarding the compound A crystallization methods (1) and (2) known in the art, the present inventors conducted experiments based on the methods described in the patent literatures.

The experiment based on the crystallization method (1) described in PTL 1 revealed a problem in that crystal precipitation was not observed, as described in Comparative Example 1 given later.

The experiment based on the crystallization method (2) described in PTLs 2 to 5 revealed a problem in that it was necessary to use a very large amount of solvent (solvent amount 20 or more times the amount of compound A on a weight basis) in order to completely dissolve compound A, as described in Comparative Example 2 given later.

It was also revealed that there was a problem in that as a result of a crystallization operation, a solid of compound A precipitated from a solution adhered in large quantities to the inner wall of a crystallization apparatus. If such a problem occurs in industrial production, the solid cannot be discharged from the apparatus, leading to a significantly low yield, and in addition, continuous production cannot be sustained. It may take time to carry out a cleaning operation for removing the adhering solid, or a highly hazardous operation such as a manual cleaning operation by operators entering inside the production apparatus may be required.

These studies have revealed that the solid of compound A known in the art with such characteristics and the crystallization method (2) known in the art have the above-described problems and thus are unsuitable for industrial production or very inefficient.

An object of the present invention is to provide means for solving the above-described problems associated with compound A.

### Solution to Problem

To solve the above-described problems, the present inventors have conducted intensive studies on how to crystallize compound A and found that as described later, a crystallization method using toluene or cyclohexane as a solvent can solve the problems associated with the crystallization methods known in the art, and a crystal obtained by this method (“crystal II”, “crystal II-a”, or “crystal II-b” described later) can solve the problem associated with the solids of compound A known in the art, thereby completing the present invention.

However, it has been found that the crystal obtained by the above method have other problems in that it is a crystal that contains much solvent even after being heated and dried under reduced pressure, and that it is inferred to be a solvated crystal, and the solvent contained in the crystal cannot be removed without melting, but melting compromises the easy-to-handle powder form.

Thus, the present inventors have conducted further studies and found that a particular crystal and a method for producing the crystal can solve the above-described problems while also solving the other problems, thereby completing the present invention.

The present invention is as follows.
1. A crystal of a trisphenol compound represented by chemical formula (A), having an endothermic peak with a peak top temperature in a range of 168° C. to 180° C., the endothermic peak being determined by differential scanning calorimetry.

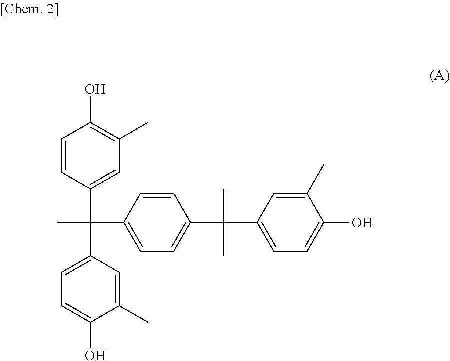 [SMILES: Cc1cc(C(C)(C)c2ccc(C(C)(c3ccc(O)c(C)c3)c3ccc(O)c(C)c3)cc2)ccc1O]

2. The crystal according to 1., having a loose bulk density in a range of 0.3 to 0.8 g/cm³.
3. The crystal according to 1., wherein an organic solvent content in the crystal is 10 wt % or less.
4. The crystal according to 1., having a purity in a range of 90% to 100%, the purity being determined by liquid chromatography analysis and expressed in area %.
5. A method for producing the crystal according to 1., including performing crystallization using a solution containing the trisphenol compound represented by chemical formula (A) and an alkylbenzene solvent having a total of 8 to 10 carbon atoms.
6. A method for producing the crystal according to 1., including performing crystallization by mixing a solution containing the trisphenol compound represented by chemical formula (A) and a carboxylic acid ester solvent having a total of 4 to 8 carbon atoms with a chain aliphatic hydrocarbon solvent having 5 to 10 carbon atoms.
7. A method for producing the crystal according to 1., including performing crystallization by mixing a solution containing the trisphenol compound represented by chemical formula (A) and an alcohol solvent having 1 to 4 carbon atoms with water.
8. A method for producing the crystal according to 1., including heating a crystal of the trisphenol compound represented by chemical formula (A) that has one endothermic peak with a peak top temperature in a range of 76° C. to 86° C. and one endothermic peak with a peak top temperature in a range of 168° C. to 180° C., the endothermic peaks being determined by differential scanning calorimetry, to 30° C. or higher and 170° C. or lower.
9. A method for producing the crystal according to 1., including melting a crystal of the trisphenol compound represented by chemical formula (A) that has peaks at diffraction angles 2θ of 14.8°±0.2°, 15.2°±0.2°, 17.5°±0.2°, and 18.1°±0.2° in a powder X-ray diffraction peak pattern obtained using Cu-Kα radiation to distill off toluene, and performing cooling.
10. A method for producing the crystal according to 1., including melting a crystal of the trisphenol compound represented by chemical formula (A) that has peaks at diffraction angles 2θ of 11.4°±0.2°, 16.6°±0.2°, and 17.8°±0.2° in a powder X-ray diffraction peak pattern obtained using Cu-Kα radiation to distill off cyclohexane, and performing cooling.
11. A crystal of a trisphenol compound represented by chemical formula (A), having peaks at diffraction angles 2θ of 13.9°±0.2°, 15.8°±0.2°, and 17.0°±0.2° in a powder X-ray diffraction peak pattern obtained using Cu-Kα radiation.

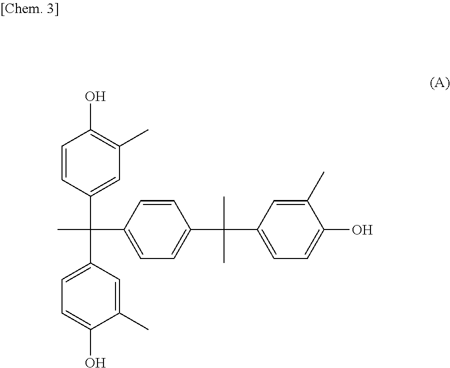 [SMILES: Cc1cc(C(C)(C)c2ccc(C(C)(c3ccc(O)c(C)c3)c3ccc(O)c(C)c3)cc2)ccc1O]

12. The crystal according to 11, having a loose bulk density in a range of 0.3 to 0.5 g/cm³.
13. The crystal according to 11, wherein an organic solvent content in the crystal is 5 wt % or less.
14. The crystal according to 11, having a purity in a range of 90% to 100%, the purity being determined by liquid chromatography analysis and expressed in area %.
15. A method for producing the crystal according to 11, including performing crystallization using a solution containing the trisphenol compound represented by chemical formula (A) and an alkylbenzene solvent having a total of 8 to 10 carbon atoms to precipitate the crystal with a temperature of the solution being in a range of 50° C. to 75° C.
16. A method for producing the crystal according to 11, including performing crystallization by mixing a solution containing the trisphenol compound represented by chemical formula (A) and a carboxylic acid ester solvent having a total of 4 to 8 carbon atoms with a chain aliphatic hydrocarbon solvent having 5 to 10 carbon atoms to precipitate the crystal with a temperature of the solution being in a range of 50° C. to 110° C.
17. A method for producing the crystal according to 11, including heating a crystal of the trisphenol compound represented by chemical formula (A) that has peaks at diffraction angles 2θ of 13.9°±0.2°, 15.0°±0.2°, 15.8°±0.2°, and 17.0°±0.2° in a powder X-ray diffraction peak pattern obtained using Cu-Kα radiation to 30° C. or higher and 170° C. or lower.
18. A method for producing the crystal according to 11, including melting a crystal of the trisphenol compound represented by chemical formula (A) that has peaks at diffraction angles 2θ of 14.8°±0.2°, 15.2°±0.2°, 17.5°±0.2°, and 18.1°±0.2° in a powder X-ray diffraction peak pattern obtained using Cu-Kα radiation to distill off toluene, and performing cooling.
19. A method for producing the crystal according to 11, including melting a crystal of the trisphenol compound represented by chemical formula (A) that has peaks at diffraction angles 2θ of 11.4°±0.2°, 16.6°±0.2°, and 17.8°±0.2° in a powder X-ray diffraction peak pattern obtained using Cu-Kα radiation to distill off cyclohexane, and performing cooling.
20. A crystal of a trisphenol compound represented by chemical formula (A), having peaks at diffraction angles 2θ of 13.9°±0.2°, 15.0°±0.2°, 15.8°±0.2°, and 17.0°±0.2° in a powder X-ray diffraction peak pattern obtained using Cu-Kα radiation.

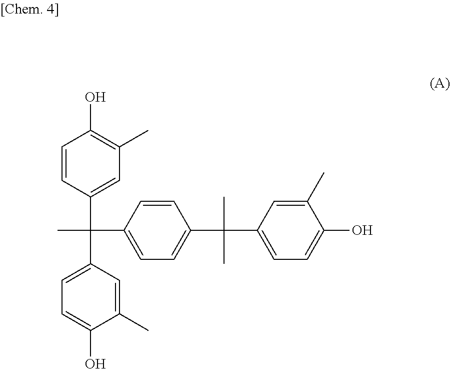 [SMILES: Cc1cc(C(C)(C)c2ccc(C(C)(c3ccc(O)c(C)c3)c3ccc(O)c(C)c3)cc2)ccc1O]

21. A method for producing the crystal according to 20., including performing crystallization by mixing a solution containing the trisphenol compound represented by chemical formula (A) and methanol with water.
22. A crystal of a trisphenol compound represented by chemical formula (A), having peaks at diffraction angles 2θ of 11.4°±0.2°, 14.0°±0.2°, and 16.3°±0.2° in a powder X-ray diffraction peak pattern obtained using Cu-Kα radiation.

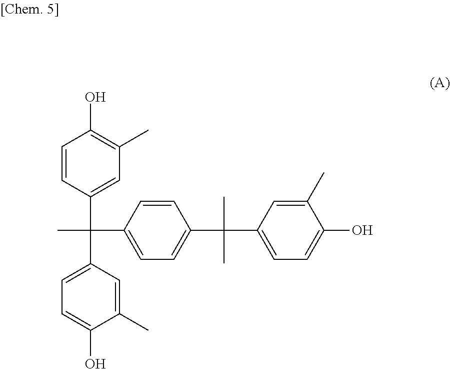 [SMILES: Cc1cc(C(C)(C)c2ccc(C(C)(c3ccc(O)c(C)c3)c3ccc(O)c(C)c3)cc2)ccc1O]

23. The crystal according to 22., having a loose bulk density in a range of 0.4 to 0.8 g/cm³.
24. The crystal according to 22., wherein an organic solvent content in the crystal is 5 wt % or less.
25. The crystal according to 22., having a purity in a range of 90% to 100%, the purity being determined by liquid chromatography analysis and expressed in area %.
26. A method for producing the crystal according to 22., including performing crystallization using a solution containing the trisphenol compound represented by chemical formula (A) and an alkylbenzene solvent having a total of 8 to 10 carbon atoms to precipitate the crystal with a temperature of the solution being in a range of 10° C. to 45° C.
27. A method for producing the crystal according to 22., including performing crystallization by mixing a solution containing the trisphenol compound represented by chemical formula (A) and a carboxylic acid ester solvent having a total of 4 to 8 carbon atoms with a chain aliphatic hydrocarbon solvent having 5 to 10 carbon atoms to precipitate the crystal with a temperature of the solution being in a range of 10° C. to 45° C.
28. A crystal of a trisphenol compound represented by chemical formula (A), having an endothermic peak with a peak top temperature in a range of 103° C. to 116° C., the endothermic peak being determined by differential scanning calorimetry.

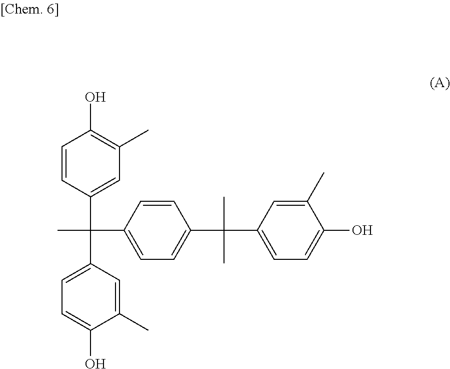 [SMILES: Cc1cc(C(C)(C)c2ccc(C(C)(c3ccc(O)c(C)c3)c3ccc(O)c(C)c3)cc2)ccc1O]

29. The crystal according to 28., having peaks at diffraction angles 2θ of 14.8°±0.2°, 15.2°±0.2°, 17.5°±0.2°, and 18.1°±0.2° in a powder X-ray diffraction peak pattern obtained using Cu-Kα radiation.
30. A method for producing the crystal according to 28. or 29., including performing crystallization using a solution containing the trisphenol compound represented by chemical formula (A) and toluene.
31. The crystal according to 28., having peaks at diffraction angles 2θ of 11.4°±0.2°, 16.6°±0.2°, and 17.8°±0.2° in a powder X-ray diffraction peak pattern obtained using Cu-Kα radiation.
32. The crystal according to 31., having a loose bulk density in a range of 0.3 to 0.6 g/cm³.
33. A method for producing the crystal according to 28. or 31., including performing crystallization using a solution containing the trisphenol compound represented by chemical formula (A) and cyclohexane.

### Advantageous Effects of Invention

The crystal of compound A according to the present invention can achieve a reduction in the amount of solvent used and does not cause a problem of adhering in large quantities to the inner wall of a crystallization apparatus in producing compound A by a crystallization operation, and thus can improve the efficiency and convenience in producing compound A and can be efficiently handled in situations in industrial utilization, such as transportation and use.

The method for producing the crystal of compound A according to the present invention can produce a crystal as described above, and can achieve a reduction in the amount of solvent used and does not cause a problem of adhering in large quantities to the inner wall of a crystallization apparatus during the process for producing the crystal, thus being a method that can efficiently produce a convenient crystal of compound A.

### BRIEF DESCRIPTION OF DRAWINGS

FIG. 1 shows a chart of differential scanning calorimetry (DSC) data of a crystal II (crystal II-a) of compound A obtained in Example 1.

FIG. 2 shows a chart of powder X-ray diffraction (PXRD) measurement of the crystal II-a (crystal II) of compound A obtained in Example 1.

FIG. 3 shows a chart of differential scanning calorimetry (DSC) data of a crystal I (crystal α) of compound A obtained in Example 2.

FIG. 4 shows a chart of powder X-ray diffraction (PXRD) measurement of the crystal α (crystal I) of compound A obtained in Example 2.

FIG. 5 shows a chart of differential scanning calorimetry (DSC) data of a crystal I (crystal α) of compound A obtained in Example 3.

FIG. 6 shows a chart of powder X-ray diffraction (PXRD) measurement of the crystal α (crystal I) of compound A obtained in Example 3.

FIG. 7 shows a chart of differential scanning calorimetry (DSC) data of a crystal I (crystal β) of compound A obtained in Example 4.

FIG. 8 shows a chart of powder X-ray diffraction (PXRD) measurement of the crystal β (crystal I) of compound A obtained in Example 4.

FIG. 9 shows a chart of differential scanning calorimetry (DSC) data of a crystal I (crystal β) of compound A obtained in Example 5.

FIG. 10 shows a chart of powder X-ray diffraction (PXRD) measurement of the crystal β (crystal I) of compound A obtained in Example 5.

FIG. 11 shows a chart of differential scanning calorimetry (DSC) data of a mixture of a crystal I (crystal β) and a crystal II (crystal II-a) of compound A obtained in Example 6.

FIG. 12 shows a chart of powder X-ray diffraction (PXRD) measurement of the mixture of the crystal β (crystal I) and the crystal II-a (crystal II) of compound A obtained in Example 6.

FIG. 13 shows a chart of differential scanning calorimetry (DSC) data of a crystal I (a mixture of a crystal α and a crystal β) of compound A obtained in Example 7.

FIG. 14 shows a chart of powder X-ray diffraction (PXRD) measurement of the mixture (crystal I) of the crystal α and the crystal β of compound A obtained in Example 7.

FIG. 15 shows a chart of differential scanning calorimetry (DSC) data of a crystal I (crystal α) of compound A obtained in Example 8.

FIG. 16 shows a chart of powder X-ray diffraction (PXRD) measurement of the crystal α (crystal I) of compound A obtained in Example 8.

FIG. 17 shows a chart of differential scanning calorimetry (DSC) data of a crystal I-a (crystal α′) of compound A obtained in Example 9.

FIG. 18 shows a chart of powder X-ray diffraction (PXRD) measurement of the crystal α′ (crystal I-a) of compound A obtained in Example 9.

FIG. 19 shows a chart of differential scanning calorimetry (DSC) data of a crystal I (crystal α) of compound A obtained in Example 10.

FIG. 20 shows a chart of powder X-ray diffraction (PXRD) measurement of the crystal α (crystal I) of compound A obtained in Example 10.

FIG. 21 shows a chart of differential scanning calorimetry (DSC) data of a crystal I-a (crystal α′) of compound A obtained in Example 11.

FIG. 22 shows a chart of powder X-ray diffraction (PXRD) measurement of the crystal α′ (crystal I-a) of compound A obtained in Example 11.

FIG. 23 shows a chart of differential scanning calorimetry (DSC) data of [crystal 12-1] among crystals of compound A obtained in Example 12.

FIG. 24 shows a chart of differential scanning calorimetry (DSC) data of [crystal 12-3] among the crystals of compound A obtained in Example 12.

FIG. 25 shows a chart of differential scanning calorimetry (DSC) data of [crystal 12-4] among the crystals of compound A obtained in Example 12.

FIG. 26 shows a chart of powder X-ray diffraction (PXRD) measurement of [crystal 12-2] among the crystals of compound A obtained in Example 12.

FIG. 27 shows a chart of powder X-ray diffraction (PXRD) measurement of [crystal 12-3] among the crystals of compound A obtained in Example 12.

FIG. 28 shows a chart of powder X-ray diffraction (PXRD) measurement of [crystal 12-4] among the crystals of compound A obtained in Example 12.

FIG. 29 shows a chart of differential scanning calorimetry (DSC) data of a crystal I (crystal α) of compound A obtained in Example 13.

FIG. 30 shows a chart of powder X-ray diffraction (PXRD) measurement of the crystal α (crystal I) of compound A obtained in Example 13.

FIG. 31 shows a chart of differential scanning calorimetry (DSC) data of a crystal I (crystal β) of compound A obtained in Example 14.

FIG. 32 shows a chart of powder X-ray diffraction (PXRD) measurement of the crystal β (crystal I) of compound A obtained in Example 14.

FIG. 33 shows a chart of differential scanning calorimetry (DSC) data of a crystal I (crystal β) of compound A obtained in Example 15.

FIG. 34 shows a chart of powder X-ray diffraction (PXRD) measurement of the crystal β (crystal I) of compound A obtained in Example 15.

FIG. 35 shows a chart of differential scanning calorimetry (DSC) data of a crystal II (crystal II-b) of compound A obtained in Example 16.

FIG. 36 shows a chart of powder X-ray diffraction (PXRD) measurement of the crystal II-b (crystal II) of compound A obtained in Example 16.

FIG. 37 shows a chart of differential scanning calorimetry (DSC) data of a crystal I (crystal α) of compound A obtained in Example 17.

FIG. 38 shows a chart of powder X-ray diffraction (PXRD) measurement of the crystal α (crystal I) of compound A obtained in Example 17.

FIG. 39 shows a chart of differential scanning calorimetry (DSC) data of a crystal II (crystal II-b) of compound A obtained in Example 18.

FIG. 40 shows a chart of powder X-ray diffraction (PXRD) measurement of the crystal II-b (crystal II) of compound A obtained in Example 18.

FIG. 41 shows a chart of differential scanning calorimetry (DSC) data of a crystal of compound A obtained in Reference Example 2.

FIG. 42 shows a chart of powder X-ray diffraction (PXRD) measurement of the crystal of compound A obtained in Reference Example 2.

FIG. 43 shows a chart of differential scanning calorimetry (DSC) data of a crystal of compound A obtained in Reference Example 4.

FIG. 44 shows a chart of powder X-ray diffraction (PXRD) measurement of the crystal of compound A obtained in Reference Example 4.

### DESCRIPTION OF EMBODIMENTS

The method of synthesizing a trisphenol compound represented by chemical formula (A) according to the present invention is not particularly limited, and the trisphenol compound can be synthesized by a known method.

For example, the trisphenol compound represented by chemical formula (A) can be synthesized by reacting o-cresol and p-isopropenylacetophenone in the presence of an acid catalyst as shown by the following reaction formula.

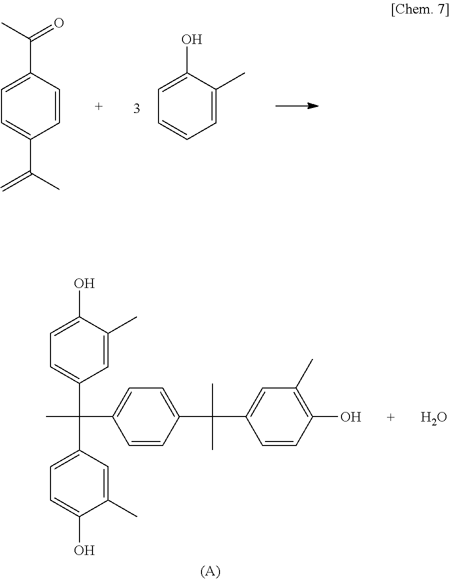

Alternatively, the trisphenol compound can be synthesized by methods described in, for example, Chinese Patent No. 107011124, Korean Patent NO. 10-1786888, and International Publication No. 2010/134559.

A reaction liquid containing the trisphenol compound represented by chemical formula (A) obtained by the above synthesis reaction can be subjected, according to a conventional method, to neutralization of the catalyst used for the reaction and to an operation of removing the starting materials used in excess of the stoichiometric amount and unnecessary components such as the catalyst and a reaction solvent by washing with water, distillation, or the like. When compound A is synthesized as shown in the above reaction formula, the starting material o-cresol is preferably used in excess of the stoichiometric amount, and in this case, the surplus o-cresol after the reaction is removed by distillation for a crystallization process described later. Alternatively, the water washing operation may be performed after an organic solvent that dissolves compound A contained in the reaction liquid and separates from water is mixed to form a solution of compound A.

Thereafter, removal of the solvent in the liquid containing compound A by a distillation operation or the like and mixing of a crystallization solvent are performed as needed, and operations such as crystallization and separation by column chromatography are performed, whereby compound A can be isolated.

The crystal of compound A according to the present invention is produced by performing a crystallization process described later using compound A in the form of, for example, a liquid obtained by treating a reaction liquid as described above, a solid obtained by a method known in the art, the crystal of the present invention or a polymorphic mixture including the crystal, or any other crystal or solid obtained by any of the methods in Comparative Examples and Reference Examples given later.

### <Crystal I>

The crystal of the trisphenol compound represented by chemical formula (A) according to the present invention has an endothermic peak with a peak top temperature in the range of 168° C. to 180° C., the endothermic peak being determined by differential scanning calorimetry. Hereinafter, such a crystal may be referred to as a “crystal I”. The peak top temperature is preferably in the range of 170° C. to 180° C., more preferably in the range of 175° C. to 180° C.

In differential scanning calorimetry analysis of the crystal of the trisphenol compound represented by chemical formula (A) according to the present invention, there are a case where one endothermic peak appears in the range of 168° C. to 180° C. and a case where two endothermic peaks, one in the range of 168° C. to 180° C. and the other in a different range, appear. In particular, the case where one endothermic peak appears is preferred.

One example of the case where two endothermic peaks appear in differential scanning calorimetry analysis is a case where one appears in the range of 168° C. to 180° C. and the other appears in the range of 76° C. to 86° C. Hereinafter, a crystal having this feature may be referred to as a “crystal I-a”. The crystal in this case is inferred to be a crystal solvated with an alcohol solvent having 1 to 4 carbon atoms, and it is inferred that the endothermic peak in the range of 76° C. to 86° C. indicates heat absorption at the time of desolvation of the alcohol solvent having 1 to 4 carbon atoms in the crystal and the endothermic peak in the range of 168° C. to 180° C. indicates heat absorption at the time of melting of the crystal that has underwent desolvation. The alcohol solvent having 1 to 4 carbon atoms is particularly preferably methanol.

The crystal of compound A according to the present invention having such a feature is preferred because it allows the solvent to be removed without melting even though it is a crystal solvated with the alcohol solvent having 1 to 4 carbon atoms, and can be used to obtain a crystal I (or a “crystal α” described later) which has a low solvent content and which is in powder form and easy to handle.

Another example of the case where two endothermic peaks appear in differential scanning calorimetry analysis is a case where one appears in the range of 103° C. to 112° C. and the other appears in the range of 168° C. to 180° C. In such a case, for example, when toluene is contained in a solution for precipitating the crystal of compound A, a “crystal II” described later may also precipitate. A mixture with the crystal of the present invention can be determined to be a mixture of two or more crystals, for example, by analyzing the crystal phase by powder X-ray diffractometry.

Such a crystal I of compound A preferably has a loose bulk density in the range of 0.3 to 0.8 g/cm³. The loose bulk density is more preferably in the range of 0.33 to 0.75 g/cm³, still more preferably in the range of 0.45 to 0.7 g/cm³, particularly preferably in the range of 0.5 to 0.7 g/cm³.

The crystal I having such a feature is preferred because it has a high bulk density sufficiently advantageous for industrial production and thus contributes to producing compound A efficiently in an easy-to-handle form.

In such a crystal I of compound A, an organic solvent content is preferably 10 wt % or less. The organic solvent content is more preferably 6 wt % or less, still more preferably 4 wt % or less, particularly preferably 2 wt % or less.

The crystal I having such a feature is preferred because it has a low solvent content and thus is suitable for preservation and transportation of compound A, the number of equivalents in producing a resin or a derivative using the crystal I can be easily optimized, and furthermore the amount of exposure to the solvent volatilized during the production can be reduced to contribute to the health of handlers and environmental conservation.

Such a crystal I of compound A preferably has a purity in the range of 90% to 100%, the purity being determined by liquid chromatography analysis and expressed in area %. The purity is more preferably in the range of 93% to 100%, still more preferably in the range of 95% to 100%, particularly preferably in the range of 98% to 100%.

Methods for producing the crystal I of compound A according to the present invention include production methods 1 to 6 as described below. Of these, production method 1, 2, 3, or 4 is preferred because it can produce a crystal of compound A that is in powder form and easy to handle.

Production method 1, 2, or 4 is more preferred because it can produce a crystal of compound A that allows the solvent contained in the crystal to be removed without melting, that has a low solvent content, and that is in powder form and easy to handle.

Production method 1 or 2 is particularly preferred because it can produce a crystal of compound A in powder form that does not solvate with the solvent in a crystallization liquid from the time of precipitation of the crystal and that allows the solvent to be removed by drying without melting or desolvation.

<Crystal I production method 1>

One method for producing the crystal I of compound A according to the present invention is a method including performing crystallization using a solution containing compound A and an alkylbenzene solvent having a total of 8 to 10 carbon atoms.

Specific examples of the alkylbenzene solvent having a total of 8 to 10 carbon atoms include ethylbenzene, para-xylene, meta-xylene, ortho-xylene (a total of 8 carbon atoms), mesitylene, 2-ethyltoluene, 3-ethyltoluene, 4-ethyltoluene (a total of 9 carbon atoms), tetralin, 1,2-diethylbenzene, 1,3-diethylbenzene, and 1,4-diethylbenzene (a total of 10 carbon atoms). Of these, alkylbenzene solvents having a total of 8 carbon atoms are preferred, and ethylbenzene is particularly preferred.

One of these alkylbenzene solvents may be used, or two or more of them may be used, in which case their content ratio is not particularly limited. What is called mixed xylene, which is a mixture of ethylbenzene, para-xylene, meta-xylene, and ortho-xylene, can also be used.

The combination of types of alkylbenzenes having a total of 8 to 10 carbon atoms is preferably a combination of alkylbenzenes selected from ethylbenzene, para-xylene, meta-xylene, and ortho-xylene, and their mixing ratio is preferably as follows: the total of para-xylene, meta-xylene, and ortho-xylene, 70 to 90 wt %; ethylbenzene, 10 to 30 wt %.

The amount of solvent used in this case is in the range of 1 to 10 times, more preferably in the range of 1.5 to 5 times, still more preferably in the range of 2 to 4 times, particularly preferably 2 to 3 times the amount of crude crystal of compound A or distillation residue on a weight basis.

In precipitating the crystal, it is not necessary to use a seed crystal, but it is preferable to use a seed crystal. The crystal used as a seed crystal is a crystal I, and a crystal precipitated without a seed crystal may be used as a seed crystal.

The crystal is precipitated with the temperature of the solution being in the range of 10° C. to 75° C. After the crystal has started to precipitate, the same temperature is maintained to increase the amount of precipitation of the crystal. The time during which the temperature is maintained is not particularly limited, but is typically in the range of 1 to 120 hours.

After the amount of precipitation of the crystal is increased, the liquid containing the crystal can be cooled, and the final cooling temperature is preferably 10° C. to 30° C. The crystal I is precipitated by the above crystallization operation. The precipitated crystal I can be isolated through separation by a filtration operation.

The crystal separated by filtration is preferably dried in order to remove the solvent used in the crystallization. The drying temperature is in the range of 25° C. to 140° C., more preferably in the range of 50° C. to 120° C. The drying may be performed under normal pressure or reduced pressure, but in the case of industrial practice, the drying is preferably performed under reduced pressure because the solvent can be removed more efficiently.

This series of operations are preferably performed in an inert gas atmosphere, such as nitrogen or argon, where the content of oxygen, which can cause oxidation or electrostatic ignition due to volatilized solvent, is low.

### <Crystal I Production Method 2>

Another method for producing the crystal I of compound A according to the present invention is a method including performing crystallization by mixing a solution containing compound A and a carboxylic acid ester solvent having a total of 4 to 8 carbon atoms with a chain aliphatic hydrocarbon solvent having 5 to 10 carbon atoms.

Specific examples of the carboxylic acid ester solvent having a total of 4 to 8 carbon atoms for use include ethyl acetate (a total of 4 carbon atoms); ethyl propionate (a total of 5 carbon atoms); butyl acetate (a total of 6 carbon atoms); amyl acetate, isoamyl acetate, 2-methylbutyl acetate (a total of 7 carbon atoms); and n-hexyl acetate (a total of 8 carbon atoms). Of these, butyl acetate is particularly preferred.

The amount of the carboxylic acid ester solvent having a total of 4 to 8 carbon atoms used is in the range of 0.3 to 5 times, more preferably in the range of 0.5 to 3 times, particularly preferably in the range of 0.5 to 2 times the amount of compound A used, on a weight basis.

The chain aliphatic hydrocarbon solvent having 5 to 10 carbon atoms may be specifically, for example, pentane, hexane, heptane, octane, or isooctane, and is preferably a chain aliphatic hydrocarbon solvent having 6 to 8 carbon atoms, more preferably hexane, heptane, octane, or isooctane, still more preferably hexane or isooctane, particularly preferably isooctane.

The amount of the chain aliphatic hydrocarbon solvent having 5 to 10 carbon atoms for use is in the range of 0.5 to 10 times, more preferably in the range of 1 to 5 times, still more preferably in the range of 1.5 to 4 times, particularly preferably 1.5 to 3 times the amount of compound A used, on a weight basis.

The temperature at which the crystal is precipitated is preferably 10° C. to 110° C. In precipitating the crystal, it is not necessary to use a seed crystal, but it is preferable to use a seed crystal. Although any crystal may be used as a seed crystal, the crystal I is preferred, and a crystal precipitated without a seed crystal may be used as a seed crystal. The final cooling temperature is preferably 10° C. to 30° C. The precipitated crystal can be isolated through separation by a filtration operation.

The crystal separated by filtration is preferably dried in order to remove the solvent used in the crystallization. The drying temperature is in the range of 25° C. to 140° C., more preferably in the range of 50° C. to 120° C. The drying may be performed under normal pressure or reduced pressure, but in the case of industrial practice, the drying is preferably performed under reduced pressure because the solvent can be removed more efficiently.

This series of operations are preferably performed in an inert gas atmosphere, such as nitrogen or argon, where the content of oxygen, which can cause oxidation or electrostatic ignition due to volatilized solvent, is low.

### <Crystal I Production Method 3>

Another method for producing the crystal I of compound A according to the present invention is a method including performing crystallization by mixing a solution containing compound A and an alcohol solvent having 1 to 4 carbon atoms with water. This method can produce a crystal I, particularly a crystal I having the feature that one endothermic peak appears in the range of 76° C. to 86° C. and another endothermic peak appears in the range of 168° C. to 180° C. in differential scanning calorimetry analysis.

Specific examples of the alcohol solvent having 1 to 4 carbon atoms for use include methanol, ethanol, 1-propanol, 2-propanol, 1-butanol, and 2-butanol. Of these, methanol is particularly preferred.

The amount of the alcohol solvent having 1 to 4 carbon atoms used is in the range of 0.5 to 5.0 times, preferably in the range of 0.5 to 4.5 times, more preferably in the range of 0.6 to 2.5 times, particularly preferably 0.6 to 1.5 times the amount of compound A used, on a weight basis.

The amount of water mixed is in the range of 0.5 to 9.5 times, preferably in the range of 0.5 to 4.5 times, more preferably in the range of 0.5 to 3.4 times, particularly preferably 0.5 to 2.4 times the amount of compound A used, on a weight basis.

The amount of solvent used is in the range of 1 to 10 times, more preferably in the range of 1.5 to 5 times, still more preferably in the range of 2 to 4 times, particularly preferably 2 to 3 times the amount of compound A used, on a weight basis.

In precipitating the crystal, it is not necessary to use a seed crystal, but it is preferable to use a seed crystal. The crystal used as a seed crystal is a crystal I, and a crystal precipitated without a seed crystal may be used as a seed crystal. The final cooling temperature is preferably 10° C. to 30° C. The precipitated crystal is separated by a filtration operation.

The crystal separated by filtration is dried at a temperature in the range of 40° C. to 70° C., more preferably in the range of 50° C. to 70° C., whereby the crystal I having the feature that one endothermic peak appears in the range of 76° C. to 86° C. and one endothermic peak appears in the range of 168° C. to 180° C. in differential scanning calorimetry analysis can be obtained. The pressure during the drying may be normal pressure or reduced pressure, but in the case of industrial practice, the drying is preferably performed under reduced pressure because the solvent can be removed more efficiently.

This series of operations are preferably performed in an inert gas atmosphere, such as nitrogen or argon, where the content of oxygen, which can cause oxidation or electrostatic ignition due to volatilized solvent, is low.

### <Crystal I Production Method 4>

The crystal I obtained as described above having the feature that one endothermic peak appears in the range of 76° C. to 86° C. and one endothermic peak appears in the range of 168° C. to 180° C. in differential scanning calorimetry analysis is heated in the range of 30° C. to 170° C., whereby the solvent used in the crystallization and contained in the crystal, that is, the alcohol solvent having 1 to 4 carbon atoms, is removed without melting the crystal, and a crystal I having an endothermic peak determined by differential scanning calorimetry in the range of 168° C. to 180° C. can be produced. The heating temperature is preferably in the range of 85° C. to 150° C., more preferably in the range of 85° C. to 120° C., particularly preferably in the range of 85° C. to 100° C.

The heating may be performed under normal pressure or reduced pressure, but in the case of industrial practice, the heating is preferably performed under reduced pressure because the solvent can be removed more efficiently.

Among the solvated alcohol solvents having 1 to 4 carbon atoms contained in the crystal, methanol is particularly preferred.

This series of operations are preferably performed in an inert gas atmosphere, such as nitrogen or argon, where the content of oxygen, which can cause oxidation or electrostatic ignition due to volatilized solvent, is low.

### <Crystal I Production Method 5>

Another method for producing the crystal I of compound A according to the present invention is a method including melting a crystal of compound A that has peaks at diffraction angles 2θ of 14.8°±0.2°, 15.2°±0.2°, 17.5°±0.2°, and 18.1°±0.2° in a powder X-ray diffraction peak pattern obtained using Cu-Kα radiation to distill off toluene, and performing cooling.

Here, the crystal of compound A that has peaks at diffraction angles 2θ of 14.8°±0.2°, 15.2°±0.2°, 17.5°±0.2°, and 18.1°±0.2° in a powder X-ray diffraction peak pattern obtained using Cu-Kα radiation is a “crystal II-a” described later.

The temperature at which the crystal II-a is melted to distill off toluene is in the range of 100° C. to 150° C. The pressure at this time is normal pressure or reduced pressure, and the melting is preferably performed under reduced pressure because toluene can be efficiently distilled off.

Compound A in a molten state from which toluene has been removed is, for example, sprayed (jetted) into cold air or dropped onto a cooling belt to solidify and subjected to melt granulation, whereby the crystal I of compound A can be produced in an easy-to-use form. The cooling rate at this time is not particularly limited.

This series of operations are preferably performed in an inert gas atmosphere, such as nitrogen or argon, where the content of oxygen, which can cause oxidation or electrostatic ignition due to volatilized solvent, is low.

### <Crystal I Production Method 6>

Another method for producing the crystal I of compound A according to the present invention is a method including melting a crystal of compound A that has peaks at diffraction angles 2θ of 11.4°±0.2°, 16.6°±0.2°, and 17.8°±0.2° in a powder X-ray diffraction peak pattern obtained using Cu-Kα radiation to distill off cyclohexane, and performing cooling.

Here, the crystal of compound A that has peaks at diffraction angles 2θ of 11.4°±0.2°, 16.6°±0.2°, and 17.8°±0.2° in a powder X-ray diffraction peak pattern obtained using Cu-Kα radiation is a “crystal II-b” described later.

The temperature at which the crystal II-b is melted to distill off cyclohexane is in the range of 100° C. to 150° C. The pressure at this time is normal pressure or reduced pressure, and the melting is preferably performed under reduced pressure because cyclohexane can be efficiently distilled off.

Compound A in a molten state from which cyclohexane has been removed is, for example, sprayed (jetted) into cold air or dropped onto a cooling belt to solidify and subjected to melt granulation, whereby the crystal I of compound A can be produced in an easy-to-use form. The cooling rate at this time is not particularly limited.

This series of operations are preferably performed in an inert gas atmosphere, such as nitrogen or argon, where the content of oxygen, which can cause oxidation or electrostatic ignition due to volatilized solvent, is low.

### <Crystal α>

The crystal of the trisphenol compound represented by chemical formula (A) according to the present invention has peaks at diffraction angles 2θ of 13.9°±0.2°, 15.8°±0.2°, and 17.0°±0.2° in a powder X-ray diffraction peak pattern obtained using Cu-Kα radiation. This crystal preferably has a peak further at a diffraction angle 2θ of 20.8°±0.2°, more preferably has peaks further at diffraction angles 2θ of 16.6°±0.2° and 25.6°±0.2°. Hereinafter, such a crystal may be referred to as a “crystal α”. As compared with a “crystal α′” described later, there is no peak at a diffraction angle 2θ of 15.0°±0.2°, and furthermore, there are no peaks at diffraction angles 2θ of 7.5°±0.2° and 22.5°±0.2°, which may be factors for distinguishing the “crystal α” from the “crystal α′”. The relative intensity of these peaks based on a peak with the greatest intensity is preferably 10 or more, more preferably 25 or more, but since the relative intensity may vary depending on the measuring apparatus and conditions or in the case of a mixture with another crystal, the crystal phase can be identified based on a standard analysis method of powder X-ray diffractometry.

The crystal α of compound A preferably has a loose bulk density in the range of 0.3 to 0.5 g/cm³. The loose bulk density is more preferably in the range of 0.3 to 0.45 g/cm³, still more preferably in the range of 0.33 to 0.45 g/cm³, particularly preferably in the range of 0.33 to 0.4 g/cm³.

The crystal α having such a feature is preferred because it has a high bulk density sufficiently advantageous for industrial production and thus contributes to producing compound A efficiently in an easy-to-handle form.

In the crystal α of compound A, an organic solvent content is preferably 5 wt % or less. The organic solvent content is more preferably 4 wt % or less, still more preferably 3 wt % or less, particularly preferably 2 wt % or less.

The crystal α having such a feature is preferred because it has a low solvent content and thus is suitable for preservation and transportation of compound A, the number of equivalents in producing a resin or a derivative using the crystal α can be easily optimized, and furthermore the amount of exposure to the solvent volatilized during the production can be reduced to contribute to the health of handlers and environmental conservation.

The crystal α of compound A preferably has a purity in the range of 90% to 100%, the purity being determined by liquid chromatography analysis and expressed in area %. The purity is more preferably in the range of 93% to 100%, still more preferably in the range of 95% to 100%, particularly preferably in the range of 98% to 100%.

The crystal α of compound A may have an endothermic peak with a peak top temperature in the range of 168° C. to 180° C., the endothermic peak being determined by differential scanning calorimetry. The peak temperature is preferably in the range of 170° C. to 180° C., more preferably in the range of 175° C. to 180° C.

Methods for producing the crystal α of compound A according to the present invention include production methods 1 to 5 as described below. Of these, production method 1, 2, or 3 is preferred because it can produce a crystal of compound A that allows the solvent contained in the crystal to be removed without melting, that has a low solvent content, and that is in powder form and easy to handle.

Of these, production method 1 or 2 is more preferred because it can produce a crystal of compound A in powder form that does not solvate with the solvent in a crystallization liquid from the time of precipitation of the crystal and that allows the solvent to be removed by drying without melting or desolvation.

### <Crystal α Production Method 1>

One method for producing the crystal α of compound A according to the present invention is a method including performing crystallization using a solution containing compound A and an alkylbenzene solvent having a total of 8 to 10 carbon atoms to precipitate the crystal with the temperature of the solution being in the range of 50° C. to 75° C.

Specific examples of the alkylbenzene solvent having a total of 8 to 10 carbon atoms include ethylbenzene, para-xylene, meta-xylene, ortho-xylene (a total of 8 carbon atoms), mesitylene, 2-ethyltoluene, 3-ethyltoluene, 4-ethyltoluene (a total of 9 carbon atoms), tetralin, 1,2-diethylbenzene, 1,3-diethylbenzene, and 1,4-diethylbenzene (a total of 10 carbon atoms). Of these, alkylbenzene solvents having a total of 8 carbon atoms are preferred, and ethylbenzene is particularly preferred.

One of these alkylbenzene solvents may be used, or two or more of them may be used, in which case their content ratio is not particularly limited. What is called mixed xylene, which is a mixture of ethylbenzene, para-xylene, meta-xylene, and ortho-xylene, can also be used.

The combination of types of alkylbenzenes having a total of 8 to 10 carbon atoms is preferably a combination of alkylbenzenes selected from ethylbenzene, para-xylene, meta-xylene, and ortho-xylene, and their mixing ratio is preferably as follows: the total of para-xylene, meta-xylene, and ortho-xylene, 70 to 90 wt %; ethylbenzene, 10 to 30 wt %.

The amount of solvent used in this case is in the range of 1 to 10 times, more preferably in the range of 1.5 to 5 times, still more preferably in the range of 2 to 4 times, particularly preferably 2 to 3 times the amount of crude crystal of compound A or distillation residue on a weight basis.

In precipitating the crystal, it is not necessary to use a seed crystal, but it is preferable to use a seed crystal. The crystal used as a seed crystal is a crystal α, and a crystal precipitated without a seed crystal may be used as a seed crystal.

The crystal is precipitated with the temperature of the solution being in the range of 50° C. to 75° C., and the temperature is preferably in the range of 50° C. to 70° C., particularly preferably in the range of 55° C. to 70° C. After the crystal has started to precipitate, the same temperature is maintained to increase the amount of precipitation of the crystal. The time during which the temperature is maintained is not particularly limited, but is typically in the range of 1 to 120 hours.

After the amount of precipitation of the crystal is increased, the liquid containing the crystal can be cooled, and the final cooling temperature is preferably 10° C. to 30° C. The precipitated crystal can be isolated through separation by a filtration operation.

The crystal separated by filtration is preferably dried in order to remove the solvent used in the crystallization. The drying temperature is in the range of 25° C. to 140° C., more preferably in the range of 50° C. to 120° C. The drying may be performed under normal pressure or reduced pressure, but in the case of industrial practice, the drying is preferably performed under reduced pressure because the solvent can be removed more efficiently.

This series of operations are preferably performed in an inert gas atmosphere, such as nitrogen or argon, where the content of oxygen, which can cause oxidation or electrostatic ignition due to volatilized solvent, is low.

### <Crystal α Production Method 2>

Another method for producing the crystal α of compound A according to the present invention is a method including performing crystallization by mixing a solution containing compound A and a carboxylic acid ester solvent having a total of 4 to 8 carbon atoms with a chain aliphatic hydrocarbon solvent having 5 to 10 carbon atoms to precipitate the crystal with the temperature of the solution being in the range of 50° C. to 110° C.

Specific examples of the carboxylic acid ester solvent having a total of 4 to 8 carbon atoms for use include ethyl acetate (a total of 4 carbon atoms); ethyl propionate (a total of 5 carbon atoms); butyl acetate (a total of 6 carbon atoms); amyl acetate, isoamyl acetate, 2-methylbutyl acetate (a total of 7 carbon atoms); and n-hexyl acetate (a total of 8 carbon atoms). Of these, butyl acetate is particularly preferred.

The amount of the carboxylic acid ester solvent having a total of 4 to 8 carbon atoms used is in the range of 0.3 to 5 times, more preferably in the range of 0.5 to 3 times, particularly preferably in the range of 0.5 to 2 times the amount of compound A used, on a weight basis.

The chain aliphatic hydrocarbon solvent having 5 to 10 carbon atoms may be specifically, for example, pentane, hexane, heptane, octane, or isooctane, and is preferably a chain aliphatic hydrocarbon solvent having 6 to 8 carbon atoms, more preferably hexane, heptane, octane, or isooctane, still more preferably hexane or isooctane, particularly preferably isooctane.

The amount of the chain aliphatic hydrocarbon solvent having 5 to 10 carbon atoms used is in the range of 0.5 to 10 times, more preferably in the range of 1 to 5 times, still more preferably in the range of 1.5 to 4 times, particularly preferably 1.5 to 3 times the amount of compound A used, on a weight basis.

In precipitating the crystal, it is not necessary to use a seed crystal, but it is preferable to use a seed crystal. Although any crystal may be used as a seed crystal, the crystal α is preferred, and a crystal precipitated without a seed crystal may be used as a seed crystal.

The crystal is precipitated with the temperature of the solution being in the range of 50° C. to 110° C., and the temperature is preferably in the range of 50° C. to 90° C., more preferably in the range of 50° C. to 75° C., particularly preferably in the range of 55° C. to 70° C. After the crystal has started to precipitate, the same temperature is maintained to increase the amount of precipitation of the crystal. The time during which the temperature is maintained is not particularly limited, but is typically in the range of 1 to 120 hours.

After the amount of precipitation of the crystal is increased, the liquid containing the crystal can be cooled, and the final cooling temperature is preferably 10° C. to 30° C. The precipitated crystal can be isolated through separation by a filtration operation.

The crystal separated by filtration is preferably dried in order to remove the solvent used in the crystallization. The drying temperature is in the range of 25° C. to 140° C., more preferably in the range of 50° C. to 120° C. The drying may be performed under normal pressure or reduced pressure, but in the case of industrial practice, the drying is preferably performed under reduced pressure because the solvent can be removed more efficiently.

This series of operations are preferably performed in an inert gas atmosphere, such as nitrogen or argon, where the content of oxygen, which can cause oxidation or electrostatic ignition due to volatilized solvent, is low.

### <Crystal α Production Method 3>

Another method for producing the crystal α of compound A according to the present invention is a method including heating a crystal of compound A that has peaks at diffraction angles 2θ of 13.9°±0.2°, 15.0°±0.2°, 15.8°±0.2°, and 17.0°±0.2° in a powder X-ray diffraction peak pattern obtained using Cu-Kα radiation to a temperature in the range of 30° C. to 170° C. This crystal of compound A to be heated is a “crystal α′” as described later.

The heating temperature is preferably in the range of 60° C. to 150° C., more preferably in the range of 80° C. to 120° C., particularly preferably in the range of 80° C. to 100° C. As a result of heating, the solvated methanol contained in the crystal is removed without melting the crystal, and the crystal α can be produced.

The heating may be performed under normal pressure or reduced pressure, but in the case of industrial practice, the heating is preferably performed under reduced pressure because the solvent can be removed more efficiently.

### <Crystal α′>

The crystal of compound A according to the present invention has peaks at diffraction angles 2θ of 13.9°±0.2°, 15.0°±0.2°, 15.8°±0.2°, and 17.0°±0.2° in a powder X-ray diffraction peak pattern obtained using Cu-Kα radiation. This crystal preferably has a peak further at a diffraction angle 2θ of 20.8°±0.2°, more preferably has peaks further at diffraction angles 2θ of 25.6°±0.2° and 22.5°±0.2°, particularly preferably has a peak further at a diffraction angle 2θ of 7.5°±0.2°. Hereinafter, such a crystal may be referred to as a “crystal α′”. The relative intensity of these peaks based on a peak with the greatest intensity is preferably 10 or more, more preferably 25 or more, but since the relative intensity may vary depending on the measuring apparatus and conditions or in the case of a mixture with another crystal, the crystal phase can be identified based on a standard analysis method of powder X-ray diffractometry.

The crystal α′ is inferred to be a crystal solvated with methanol from the analysis of the solvent contained in the crystal and differential scanning calorimetry analysis. In the differential scanning calorimetry analysis, there are one endothermic peak in the range of 168° C. to 180° C. and one endothermic peak in the range of 76° C. to 86° C. The endothermic peak appearing in the range of 76° C. to 86° C. is inferred to indicate desolvation of methanol, and the endothermic peak in the range of 168° C. to 180° C. is inferred to indicate melting of the crystal that has underwent desolvation.

The crystal α′ of compound A according to the present invention allows methanol to be desolvated without melting, and allows production of a crystal I (crystal α) which has a low solvent content and which is in powder form and easy to handle.

### <Crystal α′ Production Method>

The crystal α′ of compound A according to the present invention can be produced by performing crystallization by mixing a solution containing compound A and methanol with water.

The amount of methanol used is in the range of 0.5 to 5.0 times, preferably in the range of 0.5 to 4.5 times, more preferably in the range of 0.5 to 3.0 times, still more preferably in the range of 0.6 to 1.5 times, particularly preferably 0.6 to 1.0 times the amount of compound A used, on a weight basis.

The amount of water mixed is in the range of 0.5 to 9.5 times, preferably in the range of 0.5 to 4.5 times, more preferably in the range of 0.5 to 3.4 times, still more preferably in the range of 0.5 to 2.5 times, particularly preferably 0.5 to 1.5 times the amount of compound A used, on a weight basis.

The total amount of solvent used is in the range of 1 to 10 times, more preferably in the range of 1.5 to 5 times, still more preferably in the range of 2 to 4 times, particularly preferably 2 to 3 times the amount of compound A used, on a weight basis.

In precipitating the crystal, it is not necessary to use a seed crystal, but it is preferable to use a seed crystal. The crystal used as a seed crystal is a crystal α′, and a crystal precipitated without a seed crystal may be used as a seed crystal. The final cooling temperature is preferably 10° C. to 30° C. The precipitated crystal is separated by a filtration operation.

The crystal separated by filtration is dried at a temperature in the range of 40° C. to 70° C., more preferably in the range of 50° C. to 70° C., whereby the crystal α′ having a reduced solvent content can be obtained. The pressure during the drying may be normal pressure or reduced pressure, but in the case of industrial practice, the drying is preferably performed under reduced pressure because the solvent can be removed more efficiently.

This series of operations are preferably performed in an inert gas atmosphere, such as nitrogen or argon, where the content of oxygen, which can cause oxidation or electrostatic ignition due to volatilized solvent, is low.

### <Crystal α Production Method 4>

Another method for producing the crystal α of compound A according to the present invention is a method including melting a crystal of compound A that has peaks at diffraction angles 2θ of 14.8°±0.2°, 15.2°±0.2°, 17.5°±0.2°, and 18.1°±0.2° in a powder X-ray diffraction peak pattern obtained using Cu-Kα radiation to distill off toluene, and performing cooling.

Here, the crystal of compound A that has peaks at diffraction angles 2θ of 14.8°±0.2°, 15.2°±0.2°, 17.5°±0.2°, and 18.1°±0.2° in a powder X-ray diffraction peak pattern obtained using Cu-Kα radiation is a “crystal II-a” described later.

The temperature at which the crystal II-a is melted to distill off toluene is in the range of 100° C. to 150° C. The pressure at this time is normal pressure or reduced pressure, and the melting is preferably performed under reduced pressure because toluene can be efficiently distilled off.

Compound A in a molten state from which toluene has been removed is, for example, sprayed (jetted) into cold air or dropped onto a cooling belt to solidify and subjected to melt granulation, whereby the crystal I of compound A can be produced in an easy-to-use form. The cooling rate at this time is not particularly limited.

This series of operations are preferably performed in an inert gas atmosphere, such as nitrogen or argon, where the content of oxygen, which can cause oxidation or electrostatic ignition due to volatilized solvent, is low.

### <Crystal α Production Method 5>

Another method for producing the crystal α of compound A according to the present invention is a method including melting a crystal of compound A that has peaks at diffraction angles 2θ of 11.4°±0.2°, 16.6°±0.2°, and 17.8°±0.2° in a powder X-ray diffraction peak pattern obtained using Cu-Kα radiation to distill off cyclohexane, and performing cooling.

Here, the crystal of compound A that has peaks at diffraction angles 2θ of 11.4°±0.2°, 16.6°±0.2°, and 17.8°±0.2° in a powder X-ray diffraction peak pattern obtained using Cu-Kα radiation is a “crystal II-b” described later.

The temperature at which the crystal II-b is melted to distill off cyclohexane is in the range of 100° C. to 150° C. The pressure at this time is normal pressure or reduced pressure, and the melting is preferably performed under reduced pressure because cyclohexane can be efficiently distilled off.

Compound A in a molten state from which cyclohexane has been removed is, for example, sprayed (jetted) into cold air or dropped onto a cooling belt to solidify and subjected to melt granulation, whereby the crystal I of compound A can be produced in an easy-to-use form. The cooling rate at this time is not particularly limited.

This series of operations are preferably performed in an inert gas atmosphere, such as nitrogen or argon, where the content of oxygen, which can cause oxidation or electrostatic ignition due to volatilized solvent, is low.

### <Crystal β>

The crystal of the trisphenol compound represented by chemical formula (A) according to the present invention has peaks at diffraction angles 2θ of 11.4°±0.2°, 14.0°±0.2°, and 16.3°±0.2° in a powder X-ray diffraction peak pattern obtained using Cu-Kα radiation. This crystal preferably has peaks further at diffraction angles 2θ of 18.8°±0.2°, 19.8°±0.2°, and 20.3°±0.2°. Hereinafter, such a crystal may be referred to as a “crystal β”. The relative intensity of these peaks based on a peak with the greatest intensity is preferably 10 or more, more preferably 25 or more, but since the relative intensity may vary depending on the measuring apparatus and conditions or in the case of a mixture with another crystal, the crystal phase can be identified based on a standard analysis method of powder X-ray diffractometry.

The crystal β of compound A allows compound A to be produced as a crystal that does not solvate with the solvent in a crystallization liquid from the time of precipitation and that has a low solvent content as a result of drying, and allows compound A to be efficiently produced with the amount of desolvation-related heat, which is required in the case of other crystals (crystal I-a, crystal II-a, and crystal II-b), reduced.

The crystal β of compound A preferably has a loose bulk density in the range of 0.4 to 0.8 g/cm³. The loose bulk density is more preferably in the range of 0.45 to 0.75 g/cm³, still more preferably in the range of 0.45 to 0.7 g/cm³, particularly preferably in the range of 0.5 to 0.7 g/cm³.

The crystal β having such a feature is preferred because it has a high bulk density more sufficiently advantageous for industrial production and thus contributes to producing compound A efficiently in an easy-to-handle form.

In the crystal β of compound A, an organic solvent content is preferably 5 wt % or less. The organic solvent content is more preferably 4 wt % or less, still more preferably 3 wt % or less, particularly preferably 2 wt % or less.

The crystal β having such a feature is preferred because it has a low solvent content and thus is suitable for preservation and transportation of compound A, the number of equivalents in producing a resin or a derivative using the crystal β can be easily optimized, and furthermore the amount of exposure to the solvent volatilized during the production can be reduced to contribute to the health of handlers and environmental conservation.

The crystal β of compound A preferably has a purity in the range of 90% to 100%, the purity being determined by liquid chromatography analysis and expressed in area %. The purity is more preferably in the range of 93% to 100%, still more preferably in the range of 95% to 100%, particularly preferably in the range of 98% to 100%.

The crystal β of compound A may have an endothermic peak with a peak top temperature in the range of 168° C. to 180° C., the endothermic peak being determined by differential scanning calorimetry. The peak temperature is preferably in the range of 170° C. to 180° C., more preferably in the range of 175° C. to 180° C.

### <Crystal β Production Method 1>

One method for producing the crystal β of compound A according to the present invention is a method including performing crystallization using a solution containing compound A and an alkylbenzene solvent having a total of 8 to 10 carbon atoms to precipitate the crystal with the temperature of the solution being in the range of 10° C. to 45° C.

Specific examples of the alkylbenzene solvent having a total of 8 to 10 carbon atoms include ethylbenzene, para-xylene, meta-xylene, ortho-xylene (a total of 8 carbon atoms), mesitylene, 2-ethyltoluene, 3-ethyltoluene, 4-ethyltoluene (a total of 9 carbon atoms), tetralin, 1,2-diethylbenzene, 1,3-diethylbenzene, and 1,4-diethylbenzene (a total of 10 carbon atoms). Of these, alkylbenzene solvents having a total of 8 carbon atoms are preferred, and ethylbenzene is particularly preferred.

One of these alkylbenzene solvents may be used, or two or more of them may be used, in which case their content ratio is not particularly limited. What is called mixed xylene, which is a mixture of ethylbenzene, para-xylene, meta-xylene, and ortho-xylene, can also be used.

The combination of types of alkylbenzenes having a total of 8 to 10 carbon atoms is preferably a combination of alkylbenzenes selected from ethylbenzene, para-xylene, meta-xylene, and ortho-xylene, and their mixing ratio is preferably as follows: the total of para-xylene, meta-xylene, and ortho-xylene, 70 to 90 wt %; ethylbenzene, 10 to 30 wt %.

The amount of solvent used in this case is in the range of 1 to 10 times, more preferably in the range of 1.5 to 5 times, still more preferably in the range of 2 to 4 times, particularly preferably 2 to 3 times the amount of crude crystal and distillation residue on a weight basis.

In precipitating the crystal, it is not necessary to use a seed crystal, but it is preferable to use a seed crystal. The crystal used as a seed crystal is a crystal β, and a crystal precipitated without a seed crystal may be used as a seed crystal.

The crystal is precipitated with the temperature of the solution being in the range of 10° C. to 45° C. as described above, and the temperature is preferably in the range of 15° C. to 40° C., more preferably in the range of 20° C. to 40° C., particularly preferably in the range of 20° C. to 35° C. After the crystal has started to precipitate, the same temperature is maintained to increase the amount of precipitation of the crystal. The time during which the temperature is maintained is not particularly limited, but is typically in the range of 1 to 120 hours.

After the amount of precipitation of the crystal is increased, the liquid containing the crystal can be cooled, and the final cooling temperature is preferably 10° C. to 30° C. The precipitated crystal can be isolated through separation by a filtration operation.

The crystal separated by filtration is preferably dried in order to remove the solvent used in the crystallization. The drying temperature is in the range of 25° C. to 140° C., more preferably in the range of 50° C. to 120° C. The drying may be performed under normal pressure or reduced pressure, but in the case of industrial practice, the drying is preferably performed under reduced pressure because the solvent can be removed more efficiently.

### <Crystal β Production Method 2>

Another method for producing the crystal β of compound A according to the present invention is a method including performing crystallization by mixing a solution containing compound A and a carboxylic acid ester solvent having a total of 4 to 8 carbon atoms with a chain aliphatic hydrocarbon solvent having 5 to 10 carbon atoms to precipitate the crystal with the temperature of the solution being in the range of 10° C. to 45° C.

Specific examples of the carboxylic acid ester solvent having a total of 4 to 8 carbon atoms include ethyl acetate (a total of 4 carbon atoms); ethyl propionate (a total of 5 carbon atoms); butyl acetate (a total of 6 carbon atoms); amyl acetate, isoamyl acetate, 2-methylbutyl acetate (a total of 7 carbon atoms); and n-hexyl acetate (a total of 8 carbon atoms). Of these, butyl acetate is particularly preferred.

The amount of the carboxylic acid ester solvent having a total of 4 to 8 carbon atoms used is in the range of 0.3 to 5 times, more preferably in the range of 0.5 to 3 times, particularly preferably in the range of 0.5 to 2 times the amount of compound A used, on a weight basis.

The chain aliphatic hydrocarbon solvent having 5 to 10 carbon atoms may be specifically, for example, pentane, hexane, heptane, octane, or isooctane, and is preferably a chain aliphatic hydrocarbon solvent having 6 to 8 carbon atoms, more preferably hexane, heptane, octane, or isooctane, still more preferably hexane or isooctane, particularly preferably isooctane.

The amount of the chain aliphatic hydrocarbon solvent having 5 to 10 carbon atoms used is in the range of 0.5 to 10 times, more preferably in the range of 1 to 5 times, still more preferably in the range of 1.5 to 4 times, particularly preferably 1.5 to 3 times the amount of compound A used, on a weight basis.

In precipitating the crystal, it is not necessary to use a seed crystal, but it is preferable to use a seed crystal. Although any crystal may be used as a seed crystal, the crystal β is preferred, and a crystal precipitated without a seed crystal may be used as a seed crystal.

The crystal is precipitated with the temperature of the solution being in the range of 10° C. to 45° C. as described above, and the temperature is preferably in the range of 15° C. to 40° C., more preferably in the range of 20° C. to 40° C., particularly preferably in the range of 20° C. to 35° C. After the crystal has started to precipitate, the same temperature is maintained to increase the amount of precipitation of the crystal. The time during which the temperature is maintained is not particularly limited, but is typically in the range of 1 to 120 hours.

After the amount of precipitation of the crystal is increased, the liquid containing the crystal can be cooled, and the final cooling temperature is preferably 10° C. to 30° C. The precipitated crystal can be isolated through separation by a filtration operation.

The crystal separated by filtration is preferably dried in order to remove the solvent used in the crystallization. The drying temperature is in the range of 25° C. to 140° C., more preferably in the range of 50° C. to 120° C. The drying may be performed under normal pressure or reduced pressure, but in the case of industrial practice, the drying is preferably performed under reduced pressure because the solvent can be removed more efficiently.

This series of operations are preferably performed in an inert gas atmosphere, such as nitrogen or argon, where the content of oxygen, which can cause oxidation or electrostatic ignition due to volatilized solvent, is low.

### <Crystal II>

The crystal of the trisphenol compound represented by chemical formula (A) according to the present invention has an endothermic peak with a peak top temperature in the range of 103° C. to 116° C., the endothermic peak being determined by differential scanning calorimetry. Hereinafter, such a crystal may be referred to as a “crystal II”. The peak top temperature is preferably in the range of 107° C. to 115° C., more preferably in the range of 110° C. to 115° C., still more preferably in the range of 111° C. to 115° C.

### <Crystal II-a>

The crystal II includes a crystal having peaks at diffraction angles 2θ of 14.8°±0.2°, 15.2°±0.2°, 17.5°±0.2°, and 18.1°±0.2° in a powder X-ray diffraction peak pattern obtained using Cu-Kα radiation. This crystal preferably has peaks further at diffraction angles 2θ of 12.5°±0.2°, 13.1°±0.2°, and 13.5°±0.2°, more preferably has additional peaks further at 16.4°±0.2°, 18.9°±0.2°, and 20.5°±0.2°. Hereinafter, such a crystal may be referred to as a “crystal II-a”. The relative intensity of these peaks based on a peak with the greatest intensity is preferably 10 or more, more preferably 25 or more, but since the relative intensity varies depending on the measuring apparatus and conditions and, in the case of a mixture with another crystal, varies from that in the case of a single substance, the crystal phase can be identified based on a standard analysis method of powder X-ray diffractometry.

The crystal II-a is inferred to be a crystal solvated with toluene. The crystal II-a is very useful because it allows compound A to be obtained as a crystal and, in addition, has higher handleability and provides higher working efficiency in industrial production and use as compared with the properties of compound A known in the art.

In addition, the crystal II-a can be used to produce a crystal I or a crystal α of compound A having a low solvent content as described above by melting the crystal II-a to distill off toluene and performing cooling.

### <Crystal II-a Production Method>

The crystal II-a of compound A according to the present invention can be produced by performing crystallization using a solution containing compound A and toluene.

The amount of toluene used is in the range of 1 to 10 times, more preferably in the range of 1.5 to 5 times, still more preferably in the range of 2 to 4 times, particularly preferably in the range of 2 to 3 times the amount of compound A used, on a weight basis.

In precipitating the crystal, it is not necessary to use a seed crystal, but it is preferable to use a seed crystal. Any crystal may be used as a seed crystal. The final cooling temperature is preferably 10° C. to 30° C. The precipitated crystal is separated by a filtration operation.

This series of operations are preferably performed in an inert gas atmosphere, such as nitrogen or argon, where the content of oxygen, which can cause oxidation or electrostatic ignition due to volatilized solvent, is low.

### <Crystal II-b>

The crystal II includes a crystal having peaks at diffraction angles 2θ of 11.4°±0.2°, 16.6°±0.2°, and 17.8°±0.2° in a powder X-ray diffraction peak pattern obtained using Cu-Kα radiation. This crystal preferably has a peak further at a diffraction angle 2θ of 22.7°±0.2°. Hereinafter, such a crystal may be referred to as a “crystal II-b”. The relative intensity of these peaks based on a peak with the greatest intensity is preferably 10 or more, more preferably 25 or more, but since the relative intensity varies depending on the measuring apparatus and conditions and, in the case of a mixture with another crystal, varies from that in the case of a single substance, the crystal phase can be identified based on a standard analysis method of powder X-ray diffractometry.

The crystal II-b is inferred to be a crystal solvated with cyclohexane. The crystal II-b is very useful because it allows compound A to be obtained as a crystal and, in addition, has higher handleability and provides higher working efficiency in industrial production and use as compared with the properties of compound A known in the art.

In addition, the crystal II-b can be used to produce a crystal I or a crystal α of compound A having a low solvent content as described above by wholly or partially melting the crystal II-b to distill off cyclohexane and performing cooling.

Such a crystal II-b of compound A preferably has a loose bulk density in the range of 0.3 to 0.6 g/cm³. The loose bulk density is more preferably in the range of 0.3 to 0.5 g/cm³, still more preferably in the range of 0.3 to 0.45 g/cm³, particularly preferably in the range of 0.3 to 0.4 g/cm³. The crystal II-b having such a feature is preferred because it has a high bulk density sufficiently advantageous for industrial production and thus contributes to producing compound A efficiently in an easy-to-handle form.

### <Crystal II-b Production Method>

The crystal II-b of compound A according to the present invention can be produced by performing crystallization using a solution containing compound A and cyclohexane.

Since cyclohexane corresponds to a poor solvent with low solubility for compound A, it is preferable to use a good solvent with high solubility for compound A in combination. Examples of such a good solvent includes a carboxylic acid ester solvent having a total of 4 to 8 carbon atoms, which is preferred.

Specific examples of the carboxylic acid ester solvent having a total of 4 to 8 carbon atoms include ethyl acetate (a total of 4 carbon atoms); ethyl propionate (a total of 5 carbon atoms); butyl acetate (a total of 6 carbon atoms); amyl acetate, isoamyl acetate, 2-methylbutyl acetate (a total of 7 carbon atoms); and n-hexyl acetate (a total of 8 carbon atoms). Of these, butyl acetate is particularly preferred.

The amount of the good solvent (carboxylic acid ester solvent having a total of 4 to 8 carbon atoms) used is in the range of 0.3 to 5 times, more preferably in the range of 0.5 to 3 times, particularly preferably in the range of 0.5 to 2 times the amount of compound A used, on a weight basis.

The amount of cyclohexane used is in the range of 1 to 10 times, more preferably in the range of 1.5 to 5 times, still more preferably in the range of 1.5 to 4 times, particularly preferably in the range of 1.5 to 3 times the amount of compound A used, on a weight basis.

The total amount of solvent used is in the range of 1.3 to 10 times, more preferably in the range of 1.5 to 5 times, still more preferably in the range of 2 to 4 times, particularly preferably in the range of 2 to 3 times the amount of compound A used, on a weight basis.

In precipitating the crystal, it is not necessary to use a seed crystal, but it is preferable to use a seed crystal. Any crystal may be used as a seed crystal. The final cooling temperature is preferably 10° C. to 30° C. The precipitated crystal is separated by a filtration operation.

This series of operations are preferably performed in an inert gas atmosphere, such as nitrogen or argon, where the content of oxygen, which can cause oxidation or electrostatic ignition due to volatilized solvent, is low.

### EXAMPLES

The present invention will now be described more specifically with reference to Examples.

### <Analysis Methods>

### 1. Differential Scanning Calorimetry (DSC)

An obtained solid (crystal) was analyzed by differential scanning calorimetry (DSC) under the following conditions. A desolvation temperature or a melting point was determined from a peak top temperature of an endothermic peak. The amount of heat required to remove a solvating solvent per unit weight of the crystal was calculated from the area of an endothermic peak related to desolvation of the crystal.

### [Measurement Conditions]

Apparatus: DSC7020/manufactured by Hitachi High-Tech Science Corporation
Heating rate: 10° C./min.
Measurement temperature range: 30° C. to 200° C.
Measurement atmosphere: nitrogen 50 mL/min.
Measurement sample: 3 mg

### 2. Thermogravimetric-Differential Thermal Analysis

The weight loss of an obtained solid (crystal) was analyzed with a thermogravimetric-differential thermal analyzer (TG/DTA) under the following operation conditions. The weight loss observed before and after an endothermic peak was noted.

### [Measurement Conditions]

Apparatus: STA-200/manufactured by Hitachi High-Tech Science Corporation
Temperature: 30° C.→250° C. (heating rate 10° C./min.)
Measurement atmosphere: open, nitrogen 100 mL/min.
Measurement sample: crystal 10 mg

### 3. Analysis of Amount of Solvent Contained in Crystal

The amount of solvent contained in an obtained solid (crystal) was analyzed under the following conditions.

### <Analysis Condition 1: Headspace-Gas Chromatography (HS-GC)>

[Gas chromatography]
Apparatus: GC-2010/manufactured by Shimadzu Corporation
Detection type: FID
Column: manufactured by GL Sciences Inc., TC-160m, inner diameter 0.25 mm, thickness 0.25 μm
Carrier gas: nitrogen
Column temperature: 40° C. (10 min.)→heated to 300° C. at 20° C./min.→300° C. (5 min.)
Vaporization chamber temperature: 300° C.
Control mode: pressure
Total flow rate: 8.5 ml/min.
Column flow rate: 0.92 ml/min.
Linear velocity: 19.9 cm/sec.
Purge flow rate: 3.0 ml/min.
Split ratio: 5.0
Detector temperature: 310° C.
Makeup flow rate: 30.0 ml/min.
Hydrogen flow rate: 40.0 ml/min.
Air flow rate; 400.0 ml/min.
   [HS sampler]
Apparatus: TurboMatrix HS40/PerkinElmer Inc.
Oven temperature: 100° C.
Needle temperature: 105° C.
Transfer temperature: 105° C.
Temperature holding time: 20 min.
Pressing time: 3.0 min.
Withdrawal time: 0.5 min.
Injection time: 0.05 min.
GC cycle time: 44.0 min.
HS carrier gas pressure: 154.0 kPa
Headspace mode: constant
Vial penting: OFF
   <Measurement condition 2: high-performance liquid chromatography (HPLC)>
Apparatus: Shimadzu HPLC LC-20 series/manufactured by Shimadzu Corporation
Pump: LC-20AT
Column oven: CTO-20A
Detector: SPD-20A
Column: Shim-Pack CLC-ODS, 6 mm×15 cm/manufactured by Shimadzu GLC Ltd.
Oven temperature: 50° C.
Flow rate: 1.0 mL/min.
Mobile phase: (A) 0.2 vol % aqueous acetic acid solution, (B) methanol
Gradient conditions: (A) vol % (time from start of analysis) 50% (0 min.)→100% (30 min.)→100% (45 min.)
Sample injection volume: 20 μL
Detection wavelength: 280 nm, 254 nm
   4. Analysis of amount of solid adhering to flask after filtration operation

To a container after a filtration operation, a solvent used for a crystallization operation was added to wash away the remaining crystallization liquid. Methanol was added to the container, and stirring was performed at room temperature until all the solid adhering in the container dissolved. The resulting solution was analyzed by high-performance liquid chromatography to quantitatively determine the concentration of compound A, and the amount of compound A that had adhered in the container was calculated.

5. Powder X-ray diffraction analysis (PXRD)

An obtained solid (crystal) in an amount of 0.1 g was loaded in a sample loading section of a glass test plate and subjected to powder X-ray diffraction analysis using the following apparatus under the following conditions.

[Measurement conditions]
Apparatus: MiniFlex600-C/manufactured by Rigaku Corporation
X-ray source: CuKα
Scan axis: 2θ/θ
Mode: continuous
Measurement range: 2θ=5° to 90°
Step: 0.02°
Speed measurement time: 10°/min.
Entrance slit: 0.25° Receiving slit: 13.00 mm
Tube voltage: 40 kV
Tube current: 15 mA
   6. Measurement of bulk density

The value of the loose bulk density of each of the crystals obtained in Examples was calculated by dividing, by the value of the volume measured immediately after the crystal was filled in a graduated cylinder, the value of the weight of the filled crystal.

The value of the tapped bulk density of each of the crystals obtained in Examples was calculated by dividing, by the value of the volume measured after the crystal was filled in a graduated cylinder and then manually vibrated 300 times, the value of the weight of the filled crystal.

### <Comparative Example 1> Crystallization Experiment by Compound a Crystallization Method (1) Known in the Art

A crystallization experiment based on the description of Examples 3 and 4 of PTL 1, each describing a crystallization method using a mixed solvent of methyl isobutyl ketone and toluene, was performed.

A crystal I·α (3.03 g) obtained in Example 1 described later was used as compound A. This crystal, o-cresol (4.23 g), methanol (0.30 g), methyl isobutyl ketone (0.82 g), toluene (2.44 g), and ultrapure water (0.29 g) were added to a 100 mL test tube and dissolved by heating to 60° C. The amount of solvent used was 2.7 times the amount of compound A on a weight basis. After the heating, stirring was performed overnight with cooling at room temperature in order to cause recrystallization from the prepared solution in the mixed solvent of methyl isobutyl ketone and toluene. However, crystal precipitation was not observed.

The results of Comparative Example 1 have demonstrated that by (1) the crystallization method using a mixed solvent of toluene and methyl isobutyl ketone among crystallization methods known in the art, crystal precipitation is not observed, and a crystal cannot be obtained.

### <Comparative Example 2> Crystallization Experiment by Compound a Crystallization Method (2) Known in the Art

A crystallization experiment based on the description of Examples of PTLs 2 to 5, each describing a crystallization method using decane as a solvent, was performed.

A crystal II (7.3 g: solid content 6.6 g) obtained in Example 1 described later was used as compound A. This crystal and decane were added to a 300 mL four-necked flask to prepare a suspension of the crystal, and the flask was purged with nitrogen, after which the suspension was heated to a liquid temperature of 173° C., and decane was added until the crystal was completely dissolved. The amount of decane used was 160.8 g, that is, a solvent amount 24.4 times the amount of compound A on a weight basis was necessary.

Thereafter, to recrystallize compound A from the prepared decane solution, cooling to 98° C. was started at a rate of 10° C./h. Soon after the cooling was started, the solvent and the target compound in oil form were separated from each other to create an oil-out state. Thereafter, the target compound in oil form adhered to the inner wall of the flask and solidified while remaining adhered.

The heater was turned off to allow natural cooling to 25° C., and then an attempt was made to separate the solid solidified while adhering to the inner wall of the flask with a Kiriyama funnel. First, the suspension containing the floating solid was filtered through a Kiriyama funnel (first filtration operation). The solid separated by filtration was washed with decane (51.7 g), and the resulting solid was air-dried on the Kiriyama funnel. However, the amount of the obtained solid was 0.3 g, and the most of the remainder remained adhering in the flask.

The adhering solid was then scraped off with a spatula, and the solid was separated by filtration through a Kiriyama funnel using decane (75.5 g) to obtain a solid (5.0 g) (second filtration operation). Thereafter, the solid separated by filtration was transferred to a 50 ml eggplant-shaped flask and dried for 2 hours under a reduced pressure of 1.0 kPa while being heated at 60° C. A dried solid in an amount of 5.0 g was obtained.

On the other hand, the amount of compound A as a solid adhering to the flask after the adhering solid was scraped off with a spatula was quantitatively determined in the above-described manner. As a result, 1.4 g of the solid content remained in the flask. The above results showed that the solid content adhering in the flask at the time point after the first filtration operation was 6.4 g, the percentage of which was 98 wt %.

From the results of Comparative Example 2, it has become clear that among the compound A crystallization methods known in the art, (2) the crystallization method using decane, even if heating to a high temperature of 173° C. is performed, requires a very large amount of solvent used, leading to much energy and time required for heating, and is very poor in volumetric efficiency in producing compound A.

Furthermore, it has become clear that the solid of compound A adheres very much to the production apparatus and cannot substantially be subjected to the filtration operations. If adhesion to the production apparatus occurs, the solid cannot be discharged from the apparatus, leading to a significantly low yield, as well as hindering continuous production. It may take time to carry out a cleaning operation for removing the adhering solid, or a highly hazardous operation such as a manual cleaning operation by operators entering inside the production apparatus may be required.

From the above, it has become clear that the solid of compound A known in the art, which has the problems due to the above characteristics, and (2) the crystallization method known in the art, which has the problems due to the above phenomena, are unsuitable for industrial production or very inefficient.

### Example 1: Production of Crystal II·Crystal II-a

To a 5 L four-necked flask, o-cresol (975 g), methanol (132 g), and n-dodecyl mercaptan (50 g) were added, and under stirring at 25° C., hydrochloric acid gas was blown in until saturation was reached. A mixed solution of p-isopropenylacetophenone (322 g) and o-cresol (327 g) was added dropwise to the flask using a dropping funnel at a temperature of 26° C. to 30° C. over 3.5 hours, after which stirring was performed for 22.7 hours while the temperature was maintained.

Thereafter, at a temperature of 25° C. to 37° C., the reaction liquid was neutralized to be neutral using a 16% aqueous sodium hydroxide solution (865 g), a 75% aqueous phosphoric acid solution (4 g), 35% hydrochloric acid (8 g), and ultrapure water (22 g). The neutralized reaction liquid was heated to 113° C. to distill excess methanol. The total weight of the distillate at this time was 952 g.

To the residual liquid left after the distillation, toluene (1296 g) and ultrapure water (503 g) were added. The mixture was dissolved in an oil layer by heating to 90° C., and an aqueous layer was separated. Ultrapure water (403 g) was newly added to perform washing of the oil layer at 80° C., and an aqueous layer was separated to obtain an oil layer.

The internal liquid was heated to 165° C., and the pressure was reduced to 1.8 kPa to distill toluene and excess o-cresol. The total weight of the distillate at this time was 1878 g.

Toluene (2087 g) was added to the residual liquid left after the distillation, and the temperature of the liquid was decreased to 72° C., after which the solution was cooled to 40° C. at a rate of 10° C./h and then allowed to cool to 25° C., and a solid precipitated.

The precipitated solid was separated by filtration with a centrifugal filter, and the flask subjected to filtration and the solid separated by filtration were washed with toluene (414 g). The amount of compound A as a solid adhering to the flask after the filtration operation was quantitatively determined in the above-described manner. As a result, the solid content adhering to the flask was 8.5 g (0.9 wt %) with respect to the theoretical yield 938 g (based on p-isopropenylacetophenone).

The obtained solid (871 g) was added to a 5 L eggplant-shaped flask and dried for 4 hours under a reduced pressure of 0.4 kPa while being heated at 65° C.

Through the above operation, a solid (816 g) of compound A, which was the target compound, was obtained. The yield was 78% based on the p-isopropenylacetophenone used. The obtained solid contained 9.6% of toluene.

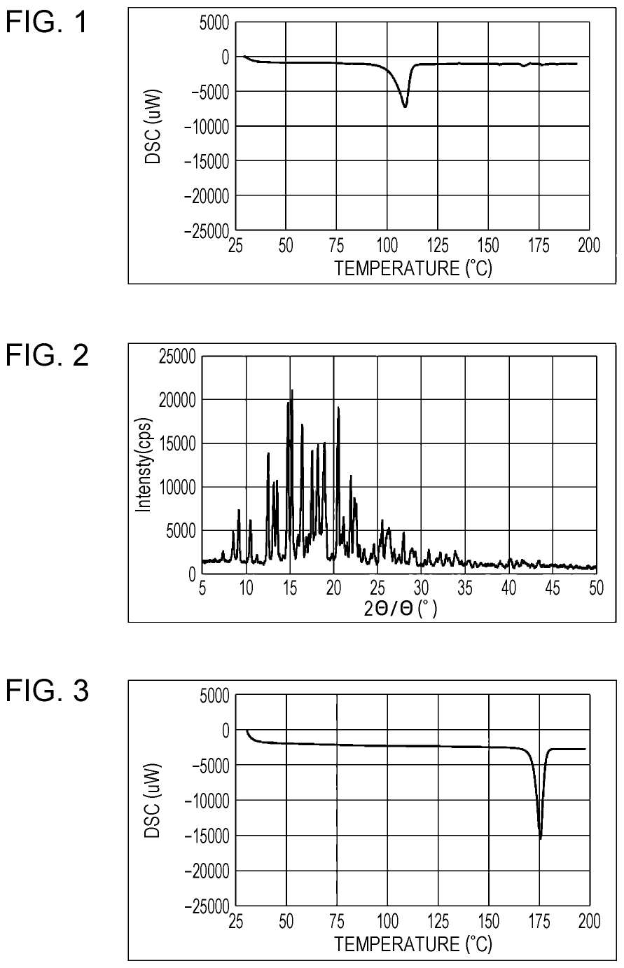

As a result of differential scanning calorimetry analysis, the obtained solid had a melting point and thus was shown to be a crystal. The melting point and the desolvation temperature were 112° C. The differential scanning calorimetry (DSC) data are shown in FIG. 1. From this analytical result, it has become clear that the obtained crystal is a crystal II.

Diffraction angles 2θ (°) of diffraction peaks appearing in powder X-ray diffraction (PXRD) measurement of the obtained crystal and peaks whose relative intensity based on a peak with the greatest intensity is 25 or more are shown in Table 1. The PXRD measurement chart is shown in FIG. 2. From this analytical result, it has become clear that the obtained crystal is a crystal II-a.

From these analytical results, it has become clear that the obtained crystal is a crystal II (crystal II-a) of compound A solvated with toluene.

TABLE 1

| | | Relative |
|---|---|---|
| No. | 2θ (°) | intensity |
| 1 | 12.5 | 47 |
| 2 | 13.2 | 35 |
| 3 | 13.5 | 38 |
| 4 | 14.8 | 76 |
| 5 | 15.2 | 72 |
| 6 | 16.4 | 61 |
| 7 | 17.5 | 47 |
| 8 | 18.1 | 63 |
| 9 | 18.9 | 100 |
| 10 | 20.5 | 63 |
| 11 | 21.9 | 33 |
| 12 | 26.3 | 44 |

### Example 2: Production of Crystal I·Crystal α

The crystal II (crystal II-a) obtained in Example 1 (33.2 g: solid content 30.0 g) was used as compound A. This crystal and ethylbenzene (75.1 g) were added to a 300 mL four-necked flask to prepare a suspension of the crystal. After the flask was purged with nitrogen, the suspension was heated to a liquid temperature of 95° C. to completely dissolve the crystal. The amount of ethylbenzene used was a solvent amount 2.5 times the amount of compound A on a weight basis.

Thereafter, to recrystallize compound A from the prepared ethylbenzene solution, the solution was cooled to 37° C. at a rate of 10° C./h. A solid started to precipitate at 75° C., and the amount of precipitating solid increased at 62° C. Thereafter, the heater was turned off to allow cooling to 25° C.

After the natural cooling to 25° C., the precipitated solid was separated by filtration with a centrifugal filter, and the flask subjected to filtration and the solid separated by filtration were washed with ethylbenzene (15.6 g). The amount of compound A as a solid adhering to the flask after the filtration operation was quantitatively determined in the above-described manner. As a result, the solid content adhering to the flask was 2.0 g (6.5 wt %) with respect to 30.0 g of the loaded solid content.

The solvent-containing solid (45.8 g) separated by filtration was added to an eggplant-shaped flask and dried for 5 hours under a reduced pressure of 0.6 kPa while being heated at 60° C.

Through the above operation, a solid (26.9 g) of compound A, which was the target compound, was obtained. The yield was 89% based on the loaded solid content. The obtained solid contained 1.4 wt % of ethylbenzene.

As a result of differential scanning calorimetry analysis, the obtained solid was found to be a crystal having a melting point of 175° C. The differential scanning calorimetry (DSC) data are shown in FIG. 3. From this analytical result, it has become clear that the obtained crystal is a crystal I.

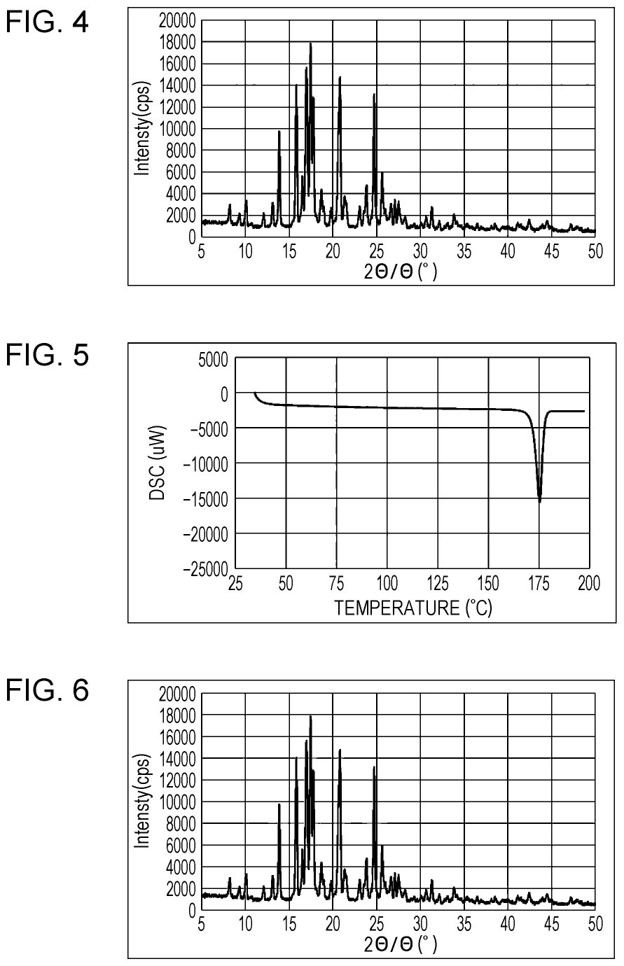

Diffraction angles 2θ (°) of diffraction peaks appearing in powder X-ray diffraction (PXRD) measurement of the obtained crystal and peaks whose relative intensity based on a peak with the greatest intensity is 25 or more are shown in Table 1. The PXRD measurement chart is shown in FIG. 4. From this analytical result, it has become clear that the obtained crystal is a crystal α.

From these analytical results, it has become clear that the obtained crystal is a crystal I (crystal α) of compound A.

TABLE 2

| | | Relative |
|---|---|---|
| No. | 2θ (°) | intensity |
| 1 | 13.9 | 50 |
| 2 | 15.8 | 69 |
| 3 | 17.0 | 100 |
| 4 | 17.4 | 92 |
| 5 | 17.7 | 66 |
| 6 | 18.7 | 33 |
| 7 | 20.6 | 63 |
| 8 | 20.8 | 51 |
| 9 | 21.3 | 31 |
| 10 | 23.8 | 27 |
| 11 | 24.7 | 62 |
| 12 | 25.6 | 26 |

### Example 3: Production of Crystal I·Crystal α

The crystal II (crystal II-a) obtained in Example 1 (33.1 g: solid content 29.9 g) was used as compound A. This crystal and ethylbenzene (75.1 g) were added to a 300 mL four-necked flask to prepare a suspension of the crystal. After the flask was purged with nitrogen, the suspension was heated to a liquid temperature of 135° C. to completely dissolve the crystal. The amount of ethylbenzene used was a solvent amount 2.5 times the amount of compound A on a weight basis.

Thereafter, to recrystallize compound A from the prepared ethylbenzene solution, the solution was cooled to 42° C. at a rate of 10° C./h. A solid started to precipitate at 70° C., and the amount of precipitating solid increased at 64° C. Thereafter, the heater was turned off to allow natural cooling to 25° C.

After the natural cooling to 25° C., the precipitated solid was separated by filtration with a centrifugal filter, and the flask subjected to filtration and the solid separated by filtration were washed with ethylbenzene (15.8 g). The amount of compound A as a solid adhering to the flask after the filtration operation was quantitatively determined in the above-described manner. As a result, the solid content adhering to the flask was 1.3 g (4.3 wt %) with respect to 29.9 g of the loaded solid content.

The solvent-containing solid (50.0 g) separated by filtration was added to an eggplant-shaped flask and dried for 6.3 hours under a reduced pressure of 0.5 kPa while being heated at 60° C.

Through the above operation, a solid (27.5 g) of compound A, which was the target compound, was obtained. The yield was 90% based on the loaded solid content. The obtained solid contained 1.8 wt % of ethylbenzene.

As a result of differential scanning calorimetry analysis, the obtained solid was found to be a crystal having a melting point of 175° C. The differential scanning calorimetry (DSC) data are shown in FIG. 5. From this analytical result, it has become clear that the obtained crystal is a crystal I.

Diffraction angles 2θ (°) of diffraction peaks appearing in powder X-ray diffraction (PXRD) measurement of the obtained crystal and peaks whose relative intensity based on a peak with the greatest intensity is 25 or more are shown in Table 3. The PXRD measurement chart is shown in FIG. 6. From this analytical result, it has become clear that the obtained crystal is a crystal α.

From these analytical results, it has become clear that the obtained crystal is a crystal I (crystal α) of compound A.

TABLE 3

| | | Relative |
|---|---|---|
| No. | 2θ (°) | intensity |
| 1 | 14.0 | 45 |
| 2 | 15.9 | 69 |
| 3 | 17.0 | 95 |
| 4 | 17.5 | 89 |
| 5 | 17.8 | 62 |
| 6 | 20.8 | 100 |
| 7 | 21.5 | 25 |
| 8 | 24.8 | 66 |
| 9 | 25.7 | 31 |

### Example 4: Production of Crystal I·Crystal β

The crystal II (crystal II-a) obtained in Example 1 (75.1 g: solid content 67.8 g) was used as compound A. This crystal was placed in an eggplant-shaped flask and heated to 135° C. under a reduced pressure of 0.6 kPa to perform a heating operation for 4.75 hours. The toluene-solvated crystal melted once at 110° C. and solidified during the heating operation at 135° C.

Thereafter, ethylbenzene (174.4 g) was added to the eggplant-shaped flask containing the solidified compound A, and the mixture was heated at 150° C. to completely dissolve the solid. The amount of ethylbenzene used was a solvent amount 2.6 times the amount of compound A on a weight basis.

Thereafter, 125.1 g of the prepared ethylbenzene solution (solid content of compound A, 34.9 g) was transferred to a 300 mL four-necked flask. After the four-necked flask was purged with nitrogen, to recrystallize compound A from the prepared ethylbenzene solution, the solution was heated to a liquid temperature of 136° C. to completely dissolve the crystal, after which the solution was cooled to 38° C. at a rate of 30° C./h. Thereafter, the heater was turned off to allow natural cooling to 25° C. During 64 hours from the start of cooling, a crystal precipitated after the natural cooling to 25° C., and the amount of precipitation increased. The precipitated solid was separated by filtration with a centrifugal filter, and the flask subjected to filtration and the solid separated by filtration were washed with ethylbenzene (17.7 g). The amount of compound A as a solid adhering to the flask after the filtration operation was quantitatively determined in the above-described manner. As a result, the solid content adhering to the flask was 1.6 g (4.6 wt %) with respect to 34.9 g of the loaded solid content.

The solvent-containing solid (34.5 g) separated by filtration was added to an eggplant-shaped flask and dried for 3.75 hours under a reduced pressure of 0.5 kPa while being heated at 60° C.

Through the above operation, a solid (32.3 g) of compound A, which was the target compound, was obtained. The yield was 91% based on the loaded solid content. The obtained solid contained 1.4 wt % of ethylbenzene.

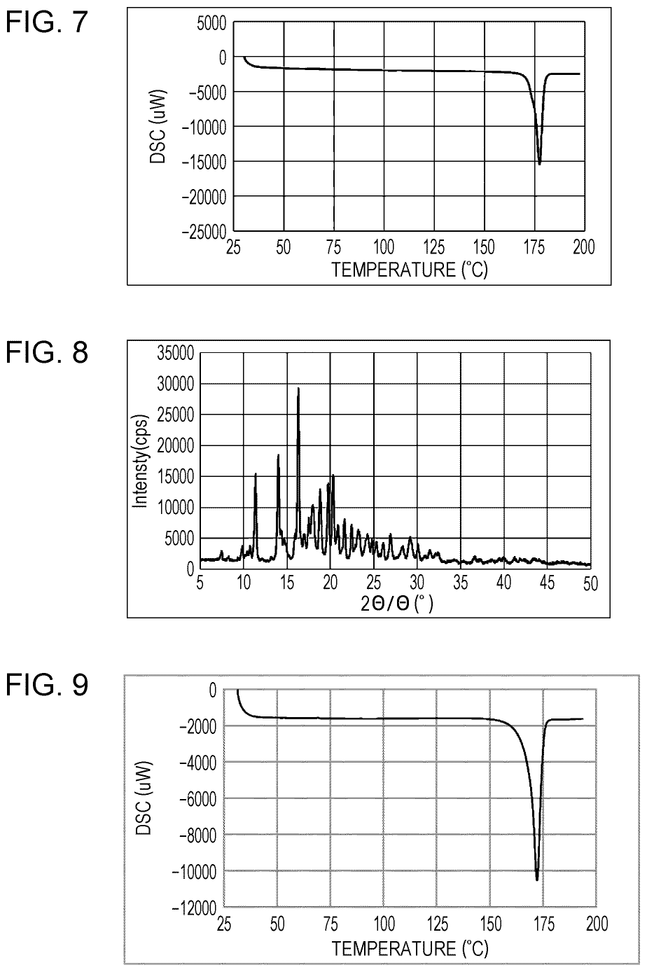

As a result of differential scanning calorimetry analysis, the obtained solid was found to be a crystal having a melting point of 177° C. The differential scanning calorimetry (DSC) data are shown in FIG. 7. From this analytical result, it has become clear that the obtained crystal is a crystal I.

Diffraction angles 2θ (°) of diffraction peaks appearing in powder X-ray diffraction (PXRD) measurement of the obtained crystal and peaks whose relative intensity based on a peak with the greatest intensity is 25 or more are shown in Table 4. The PXRD measurement chart is shown in FIG. 8. From this analytical result, it has become clear that the obtained crystal is a crystal β.

From these analytical results, it has become clear that the obtained crystal is a crystal I (crystal β) of compound A.

TABLE 4

| | | Relative |
|---|---|---|
| No. | 2θ (°) | intensity |
| 1 | 11.4 | 62 |
| 2 | 14.0 | 67 |
| 3 | 16.3 | 100 |
| 4 | 18.0 | 51 |
| 5 | 18.8 | 42 |
| 6 | 19.7 | 46 |
| 7 | 20.3 | 53 |
| 8 | 23.2 | 28 |

### Example 5: Production of Crystal I·Crystal β

To a 1 L four-necked flask, o-cresol (199.9 g), methanol (26.5 g), and n-dodecyl mercaptan (5.0 g) were added, and under stirring at 32° C., hydrochloric acid gas was blown in until saturation was reached. A mixed solution of p-isopropenylacetophenone (65.5 g) and o-cresol (67.8 g) was added dropwise to the flask using a dropping funnel at a temperature of 27° C. to 32° C. over 3 hours and 10 minutes, after which stirring was performed for 18 hours and 35 minutes while the temperature was maintained.

Thereafter, at a temperature of 25° C. to 45° C., the reaction liquid was neutralized to be neutral using a 16% aqueous sodium hydroxide solution (179.7 g), a 75% aqueous phosphoric acid solution (0.5 g), and ultrapure water (10.0 g).

Butyl acetate (137.7 g) was added. The mixture was heated to 60° C., and an aqueous layer was separated. Butyl acetate (58.1 g), isooctane (66.9 g), and ultrapure water (80.1 g) were newly added to perform washing of an oil layer at 80° C., and an aqueous layer was separated. Thereafter, ultrapure water (81.2 g) was added again to perform washing of the oil layer, and an aqueous layer was separated.

The internal liquid was heated to 140° C., and the pressure was reduced to 6.4 kPa to distill butyl acetate, isooctane, and excess o-cresol. The total weight of the distillate at this time was 415.3 g.

Butyl acetate (73.9 g) was added to the residual liquid left after the distillation to obtain a butyl acetate solution. The obtained butyl acetate solution (55.2 g) was portioned into a 300 mL eggplant-shaped flask and heated at 100° C., and the pressure was reduced to 1.0 kPa to recover excess butyl acetate. The weight of the internal liquid left after the recovery was 43.6 g.

The internal liquid was transferred to a 300 mL four-necked flask, and ethylbenzene (82.3 g) was added. A crystal I·β (0.04 g) of compound A was added at 99° C., and the solution was cooled to 26° C. at a rate of 10° C./h. Thereafter, the heater was turned off to allow natural cooling to 25° C.

The precipitated solid was separated by filtration with a centrifugal filter, and the flask subjected to filtration and the solid separated by filtration were washed with ethylbenzene (20.2 g). The amount of compound A as a solid adhering to the flask after the filtration operation was quantitatively determined in the above-described manner. As a result, the solid content adhering to the flask was 1.1 g (2.8 wt %) with respect to the theoretical yield 39.1 g (based on p-isopropenylacetophenone).

The obtained solid (42.2 g) was added to a 300 mL eggplant-shaped flask and dried for 3 hours and 15 minutes under a reduced pressure of 0.6 kPa while being heated at 60° C.

Through the above operation, a solid (27.7 g) of compound A, which was the target compound, was obtained. The yield was 69% based on the p-isopropenylacetophenone used. The obtained solid contained 0.2 wt % of butyl acetate, 1.5 wt % of ethylbenzene, and 0.5 wt % of o-cresol.

As a result of differential scanning calorimetry analysis, the obtained solid had a melting point and thus was shown to be a crystal. The melting point was 172° C. The differential scanning calorimetry (DSC) data are shown in FIG. 9. From this analytical result, it has become clear that the obtained crystal is a crystal I.

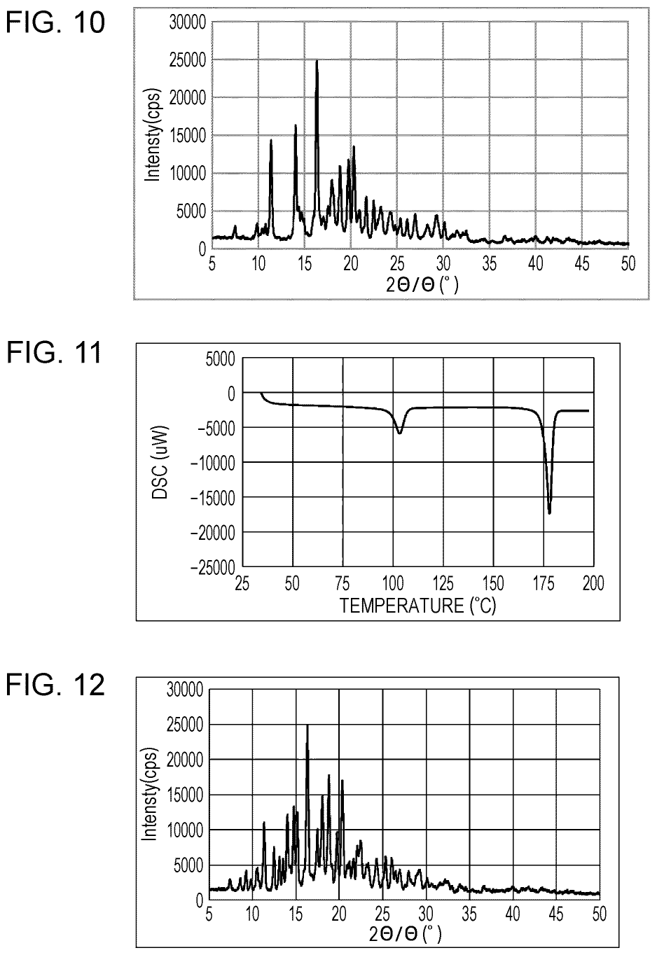

Diffraction angles 2θ (°) of diffraction peaks appearing in powder X-ray diffraction (PXRD) measurement of the obtained crystal and peaks whose relative intensity based on a peak with the greatest intensity is 25 or more are shown in Table 5. The PXRD measurement chart is shown in FIG. 10. From this analytical result, it has become clear that the obtained crystal is a crystal β.

From these analytical results, it has become clear that the obtained crystal is a crystal I (crystal β) of compound A.

TABLE 5

| | | Relative |
|---|---|---|
| No. | 2θ (°) | intensity |
| 1 | 11.3 | 61 |
| 2 | 14.0 | 61 |
| 3 | 16.3 | 100 |
| 4 | 17.9 | 43 |
| 5 | 18.8 | 41 |
| 6 | 19.7 | 44 |
| 7 | 20.3 | 52 |
| 8 | 23.2 | 30 |

### Example 6: Production of Mixture of Crystal I·Crystal β and Crystal II·Crystal II-a

The crystal II (crystal II-a) obtained in Example 1 (33.1 g: solid content 29.9 g) was used as compound A. This crystal and ethylbenzene (76.1 g) were added to a 300 mL four-necked flask to prepare a suspension of the crystal. After the flask was purged with nitrogen, the suspension was heated to a liquid temperature of 135° C. to completely dissolve the crystal. The amount of ethylbenzene used was a solvent amount 2.5 times the amount of compound A on a weight basis.

Thereafter, to recrystallize compound A from the prepared ethylbenzene solution, the solution was cooled to 25° C. at a rate of 30° C./h. During 98 hours from the start of cooling, a crystal precipitated after the natural cooling to 25° C., and the amount of precipitation increased. The precipitated solid was separated by filtration with a centrifugal filter, and the flask subjected to filtration and the solid separated by filtration were washed with ethylbenzene (16.1 g). The amount of compound A as a solid adhering to the flask after the filtration operation was quantitatively determined in the above-described manner. As a result, the solid content adhering to the flask was 1.6 g (5.5 wt %) with respect to 29.9 g of the loaded solid content.

The solvent-containing solid (41.9 g) separated by filtration was added to an eggplant-shaped flask and dried for 3.5 hours under a reduced pressure of 0.5 kPa while being heated at 60° C.

Through the above operation, a solid (25.8 g) of compound A, which was the target compound, was obtained. The yield was 82% based on the loaded solid content. The obtained solid contained 3.1 wt % of ethylbenzene and 2.2 wt % of toluene.

As a result of differential scanning calorimetry analysis, the obtained solid was found to be a crystal having endothermic peak tops at 103° C. and 178° C. The differential scanning calorimetry (DSC) data are shown in FIG. 11. From this analytical result, it has become clear that the obtained crystal is a crystal I.

Diffraction angles 2θ (°) of diffraction peaks appearing in powder X-ray diffraction (PXRD) measurement of the obtained crystal and peaks whose relative intensity based on a peak with the greatest intensity is 25 or more are shown in Table 6. The PXRD measurement chart is shown in FIG. 12. From this analytical result, it has become clear that the obtained crystal is a mixture of a crystal β and a crystal II-a.

From these analytical results, it has become clear that the obtained crystal is a mixture of a crystal I (crystal β) and a crystal II (crystal II-a) of compound A. From this, it follows that the endothermic peak top temperature at 103° C. is the melting point of the crystal II, and the endothermic peak top temperature at 178° C. is the melting point of the crystal β.

TABLE 6

| | | Relative |
|---|---|---|
| No. | 2θ (°) | intensity |
| 1 | 11.3 | 44 |
| 2 | 14.0 | 51 |
| 3 | 14.8 | 48 |
| 4 | 15.2 | 43 |
| 5 | 16.3 | 100 |
| 6 | 17.5 | 29 |
| 7 | 18.1 | 59 |
| 8 | 18.8 | 69 |
| 9 | 20.4 | 59 |
| 10 | 22.4 | 32 |

### Example 7: Production of Crystal I, Mixture of Crystal α and Crystal β

The crystal II (crystal II-a) obtained in Example 1 (33.2 g: solid content 30.0 g) was used as compound A. This crystal and mixed xylene (75.6 g) were added to a 300 mL four-necked flask to prepare a suspension of the crystal, and the flask was purged with nitrogen, after which the suspension was heated to a liquid temperature of 94° C. to completely dissolve the crystal. The amount of mixed xylene used was a solvent amount 2.5 times the amount of compound A on a weight basis.

Thereafter, to recrystallize compound A from the prepared mixed xylene solution, the solution was cooled to 35° C. at a rate of 10° C./h. A solid started to precipitate at 74° C., and the amount of precipitating solid increased at 65° C. Thereafter, the heater was turned off to allow natural cooling to 25° C.

After the natural cooling to 25° C., the precipitated solid was separated by filtration with a centrifugal filter, and the flask subjected to filtration and the solid separated by filtration were washed with mixed xylene (16.2 g). The amount of compound A as a solid adhering to the flask after the filtration operation was quantitatively determined in the above-described manner. As a result, the solid content adhering to the flask was 1.8 g (6.2 wt %) with respect to 30.0 g of the loaded solid content.

The solvent-containing solid (34.2 g) separated by filtration was added to an eggplant-shaped flask and dried for 4 hours under a reduced pressure of 1.1 kPa while being heated at 60° C.

Through the above operation, a solid (27.2 g) of compound A, which was the target compound, was obtained. The yield was 89% based on the loaded solid content. The obtained solid contained 2.1 wt % of xylene.

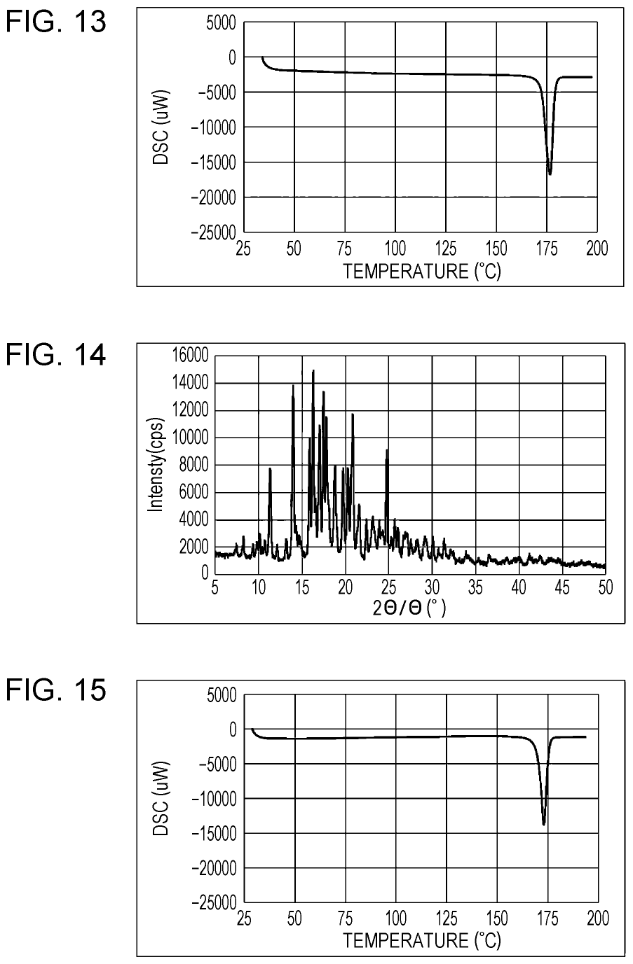

As a result of differential scanning calorimetry analysis, the obtained solid was found to be a crystal having a melting point of 177° C. The differential scanning calorimetry (DSC) data are shown in FIG. 13. From this analytical result, it has become clear that the obtained crystal is a crystal I.

Diffraction angles 2θ (°) of diffraction peaks appearing in powder X-ray diffraction (PXRD) measurement of the obtained crystal and peaks whose relative intensity based on a peak with the greatest intensity is 25 or more are shown in Table 7. The PXRD measurement chart is shown in FIG. 14. From this analytical result, it has become clear that the obtained crystal is a mixture of a crystal α and a crystal β.

TABLE 7

| | | Relative |
|---|---|---|
| No. | 2θ (°) | intensity |
| 1 | 11.3 | 57 |
| 2 | 14.0 | 82 |
| 3 | 15.9 | 59 |
| 4 | 16.3 | 100 |
| 5 | 17.0 | 78 |
| 6 | 17.5 | 69 |
| 7 | 17.8 | 78 |
| 8 | 18.8 | 53 |
| 9 | 19.8 | 47 |
| 10 | 20.3 | 53 |
| 11 | 20.9 | 69 |
| 12 | 21.6 | 42 |
| 13 | 23.1 | 30 |
| 14 | 24.7 | 35 |

### Example 8: Production of Crystal I·Crystal α by Melting Operation

As compound A, the crystal II (crystal II-a) obtained in Example 1 (199 g) was added to a 1 L eggplant-shaped flask and dried for 5 hours under a reduced pressure of 1.5 kPa while being heated at 135° C. During this process, the crystal melted into an oil, which was then cooled to obtain a solid (181 g). The obtained solid contained 0.1 wt % of toluene.

As a result of differential scanning calorimetry analysis, the obtained solid was shown to be a crystal having a melting point of 173° C. The differential scanning calorimetry (DSC) data are shown in FIG. 15. From this analytical result, it has become clear that the obtained crystal is a crystal I.

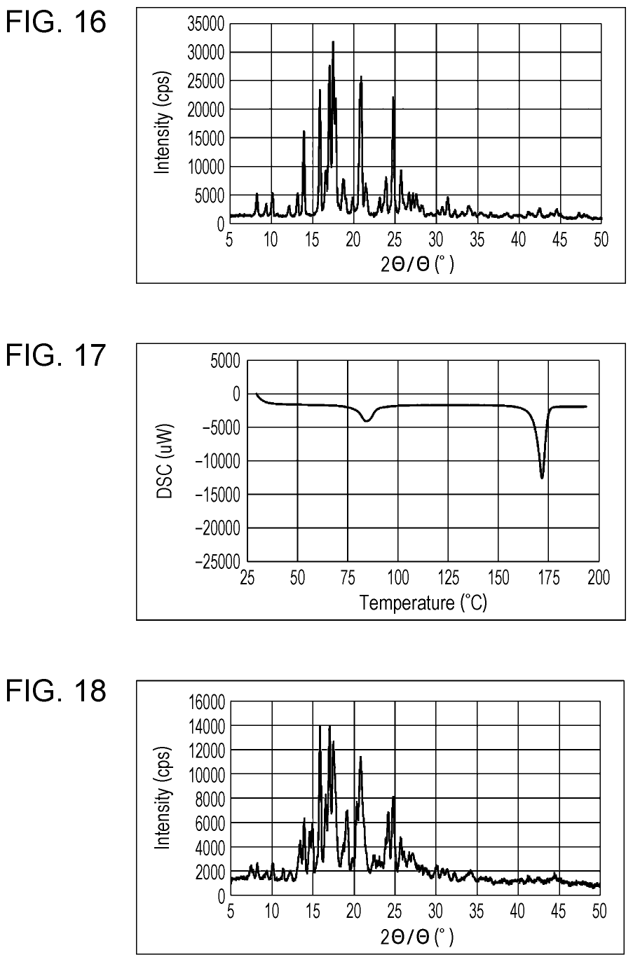

Diffraction angles 2θ (°) of diffraction peaks appearing in powder X-ray diffraction (PXRD) measurement of the obtained crystal and peaks whose relative intensity based on a peak with the greatest intensity is 25 or more are shown in Table 8. The PXRD measurement chart is shown in FIG. 16. From this analytical result, it has become clear that the obtained crystal is a crystal α.

From these analytical results, it has become clear that the obtained crystal is a crystal I (crystal α) of compound A.

TABLE 8

| | | Relative |
|---|---|---|
| No. | 2θ (°) | intensity |
| 1 | 13.9 | 46 |
| 2 | 15.9 | 70 |
| 3 | 17.0 | 100 |
| 4 | 17.5 | 96 |
| 5 | 17.8 | 75 |
| 6 | 18.7 | 26 |
| 9 | 20.7 | 74 |
| 10 | 20.9 | 43 |
| 11 | 21.4 | 27 |
| 12 | 23.9 | 28 |
| 13 | 24.7 | 75 |
| 14 | 25.7 | 32 |

### Example 9: Production of Crystal I (Crystal I-a)·Crystal α′

The crystal α (crystal I) obtained in Example 8 (13.7 g) as compound A and methanol (11.0 g) were added to a 200 mL eggplant-shaped flask and dissolved by heating to 60° C. Ultrapure water (18.8 g) was added, and the mixture was stirred overnight while being allowed to cool to 25° C. The amount of solvent used was 2.2 times the amount of compound A on a weight basis.

Since precipitation of a solid was observed in the internal liquid, the inner wall was rubbed with a spatula to promote the precipitation of the solid. After this operation, stirring was further performed overnight.

The precipitated solid was separated with a Kiriyama funnel, and the solid was washed with ultrapure water (20.1 g). After the crystallization liquid was transferred to the Kiriyama funnel, almost no solid remained in the flask, and almost no solid adhered to the inner wall of the flask. The obtained solid (18.9 g) was added to a 200 mL eggplant-shaped flask and dried for 3.5 hours under a reduced pressure of 0.6 kPa while being heated at 60° C. to obtain a solid (13.9 g).

The obtained solid contained 2.0 wt % of water and 5.2 wt % of methanol.

As a result of differential scanning calorimetry analysis, the obtained solid was shown to be a crystal having a desolvation temperature of 84° C. and a melting point of 172° C. The differential scanning calorimetry (DSC) data are shown in FIG. 17. From this analytical result, it has become clear that the obtained crystal is a crystal I-a.

Diffraction angles 2θ (°) of diffraction peaks appearing in powder X-ray diffraction (PXRD) measurement of the obtained crystal and peaks whose relative intensity based on a peak with the greatest intensity is 10 or more are shown in Table 9. The PXRD measurement chart is shown in FIG. 18. From this analytical result, it has become clear that the obtained crystal is a crystal α′.

From these analytical results, it has become clear that the obtained crystal is a crystal I-a (crystal α′) of compound A.

TABLE 9

| No. | 2θ (°) | Relative intensity |
|---|---|---|
| 1 | 13.4 | 29 |
| 2 | 13.9 | 33 |
| 3 | 14.6 | 24 |
| 4 | 14.9 | 26 |
| 5 | 15.9 | 75 |
| 6 | 16.5 | 39 |
| 7 | 17.0 | 79 |
| 8 | 17.5 | 27 |
| 9 | 17.6 | 100 |
| 10 | 18.6 | 11 |
| 11 | 19.1 | 48 |
| 12 | 20.3 | 27 |
| 13 | 20.8 | 81 |
| 14 | 21.1 | 62 |
| 15 | 23.8 | 10 |
| 16 | 24.2 | 24 |
| 17 | 24.7 | 40 |
| 18 | 25.6 | 16 |
| 19 | 26.9 | 27 |
| 20 | 34.2 | 20 |
| 21 | 35.9 | 11 |

### Example 10: Production of Crystal I·Crystal α

The crystal α′ (crystal I-a) of compound A obtained in Example 9 (5.0 g) was added to a 50 mL eggplant-shaped flask and dried for 3.25 hours under a reduced pressure of 1.6 kPa while being heated at 85° C. During this process, the crystal did not melt and remained in powder form.

Through the above operation, a crystal (4.8 g) containing 0.7 wt % of water was obtained.

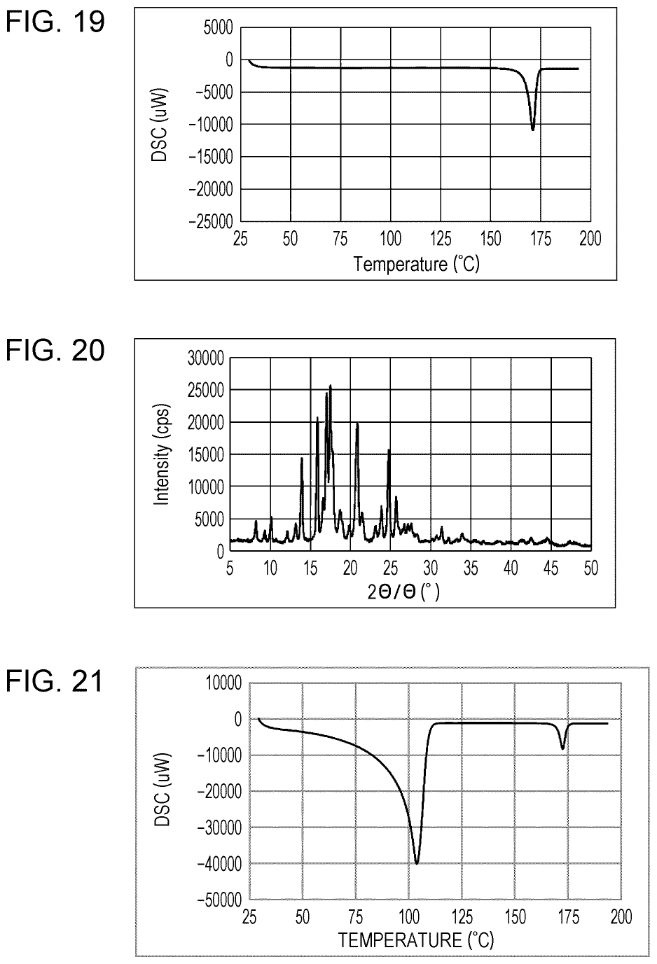

As a result of differential scanning calorimetry analysis, the obtained solid was shown to be a crystal having a melting point of 171° C. The differential scanning calorimetry (DSC) data are shown in FIG. 19. From this analytical result, it has become clear that the obtained crystal is a crystal I.

Diffraction angles 2θ (°) of diffraction peaks appearing in powder X-ray diffraction (PXRD) measurement of the obtained crystal and peaks whose relative intensity based on a peak with the greatest intensity is 10 or more are shown in Table 10. The PXRD measurement chart is shown in FIG. 20. In this analytical result, peaks at diffraction angles 2θ of 14.9°±0.2° and 22.5°±0.2°, which are peaks appearing in the crystal α′ obtained in Example 9, are absent, and these peaks are considered to be factors for distinguishing a “crystal α” from a “crystal α”. It has become clear that the obtained crystal is a crystal α.

From these analytical results, it has become clear that the obtained crystal is a crystal I (crystal α) of compound A.

TABLE 10

| No. | 2θ (°) | Relative intensity |
|---|---|---|
| 1 | 8.3 | 14 |
| 2 | 10.1 | 16 |
| 3 | 13.2 | 13 |
| 4 | 13.9 | 52 |
| 5 | 15.9 | 73 |
| 6 | 16.6 | 23 |
| 7 | 17.0 | 100 |
| 8 | 17.5 | 87 |
| 9 | 17.8 | 81 |
| 10 | 18.8 | 36 |
| 11 | 20.7 | 52 |
| 12 | 20.9 | 58 |
| 13 | 21.4 | 30 |
| 14 | 23.1 | 11 |
| 15 | 23.9 | 24 |
| 16 | 24.8 | 60 |
| 17 | 25.7 | 30 |
| 18 | 27.5 | 19 |
| 19 | 31.3 | 11 |
| 20 | 33.9 | 11 |
| 21 | 44.4 | 13 |

### Example 11: Production of Crystal I (Crystal I-a)·Crystal α′

The crystal II (crystal II-a) obtained in Example 1 (33.4 g: solid content 30.3 g) was used as compound A. This crystal was placed in an eggplant-shaped flask and heated to 135° C. under a reduced pressure of 0.6 kPa to perform a heating operation for 2 hours.

Thereafter, methanol (31.5 g) was added to the eggplant-shaped flask containing the solidified compound A, and the mixture was heated at 45° C. to completely dissolve the solid. The amount of methanol used was a solvent amount 1.0 times the amount of compound A on a weight basis.

Thereafter, 61.4 g of the prepared methanol solution (solid content 30.1 g) was transferred to a 300 mL four-necked flask. After the four-necked flask was purged with nitrogen, the temperature was raised to 57° C., and while the solution was stirred with the temperature of the solution being in the range of 57° C. to 59° C., ultrapure water (46.7 g) was added over 1 hour and 30 minutes. The amount of solvent used for the crystallization operation was 2.6 times the amount of compound A on a weight basis.

Thereafter, the solution was cooled to 26° C. at a rate of 9° C./h. Thereafter, the heater was turned off to allow natural cooling to 25° C. After the cooling, stirring was continued for 39 hours, at which point it was confirmed that a solid precipitated and dispersed in the solution.

After the slurry containing the precipitated solid was poured into a centrifugal filter, the solid in the flask was poured into the centrifugal filter using ultrapure water (75.1 g), and the solid was separated by filtration. The amount of compound A as a solid adhering to the flask after the filtration operation was quantitatively determined in the above-described manner. As a result, the solid content adhering to the flask was 1.2 g (3.9 wt %) with respect to 30.1 g of the loaded solid content. As a result of differential scanning calorimetry analysis of the obtained solid, an endothermic peak temperature attributed to evaporation of the attached solvent and desolvation was observed at 104° C., and a melting point was observed at 172° C. The differential scanning calorimetry (DSC) data are shown in FIG. 21. From this analytical result, it has become clear that the obtained crystal is a crystal I, and it has been suggested that it is a crystal I-a.

Diffraction angles 2θ (°) of diffraction peaks appearing in powder X-ray diffraction (PXRD) measurement of the obtained crystal and peaks whose relative intensity based on a peak with the greatest intensity is 10 or more are shown in Table 11. The PXRD measurement chart is shown in FIG. 22. From this analytical result, it has become clear that the obtained crystal is a crystal α′.

From these analytical results, it has become clear that the obtained crystal is a crystal I (I-a) (crystal α′) of compound A.

TABLE 11

| No. | 2θ (°) | Relative intensity |
|---|---|---|
| 1 | 7.5 | 18 |
| 2 | 13.6 | 14 |
| 3 | 14.0 | 15 |
| 4 | 14.6 | 13 |
| 5 | 15.0 | 100 |
| 6 | 15.9 | 71 |
| 7 | 16.6 | 16 |
| 8 | 17.1 | 51 |
| 9 | 17.6 | 19 |
| 10 | 17.8 | 11 |
| 11 | 19.2 | 27 |
| 12 | 20.7 | 16 |
| 13 | 20.9 | 21 |
| 14 | 21.3 | 44 |
| 15 | 22.5 | 33 |
| 16 | 24.2 | 13 |
| 17 | 25.7 | 20 |
| 18 | 27.0 | 12 |
| 19 | 28.9 | 12 |
| 20 | 30.1 | 14 |
| 21 | 34.3 | 14 |

### Example 12: Production of Crystal I (Crystal I-a)·Crystal α

The crystal α′ (crystal I-a) of compound A obtained in Example 11 (63.9 g) was added to a 300 mL eggplant-shaped flask and dried under a reduced pressure of 0.7 kPa for a total of 6.5 hours, at 30° C. for 0.5 hours, at 40° C. for 1 hour, at 40° C. to 60° C. for 0.5 hours, and at 60° C. for 4.5 hours, while portions were sampled along the way. During this process, the crystal did not melt and remained in powder form.

Crystals sampled during the drying operation were as follows.
[Crystal 12-1] At point of drying temperature of 40° C. and 1 hour and 30 minutes from start of drying
[Crystal 12-2] At point of drying temperature of 45° C. and 1 hour and 40 minutes from start of drying
[Crystal 12-3] At point of drying temperature of 60° C. and 3 hours from start of drying
[Crystal 12-4] At point of completion of drying

Through the above operation, a crystal [crystal 12-4] (26.3 g) containing 0.5 wt % of methanol and 0.2 wt % of water was obtained.

(Differential scanning calorimetry analysis result)

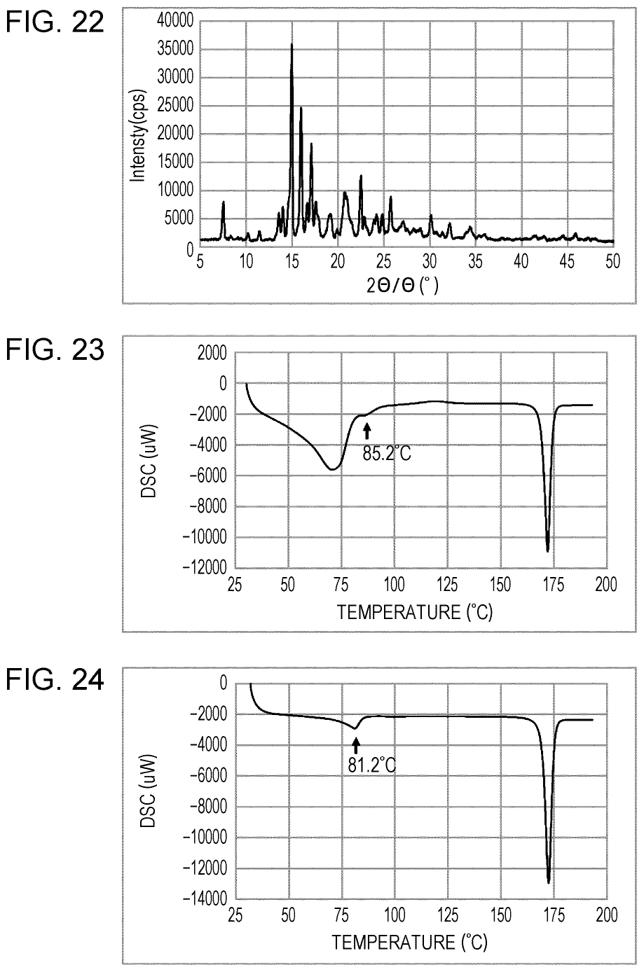

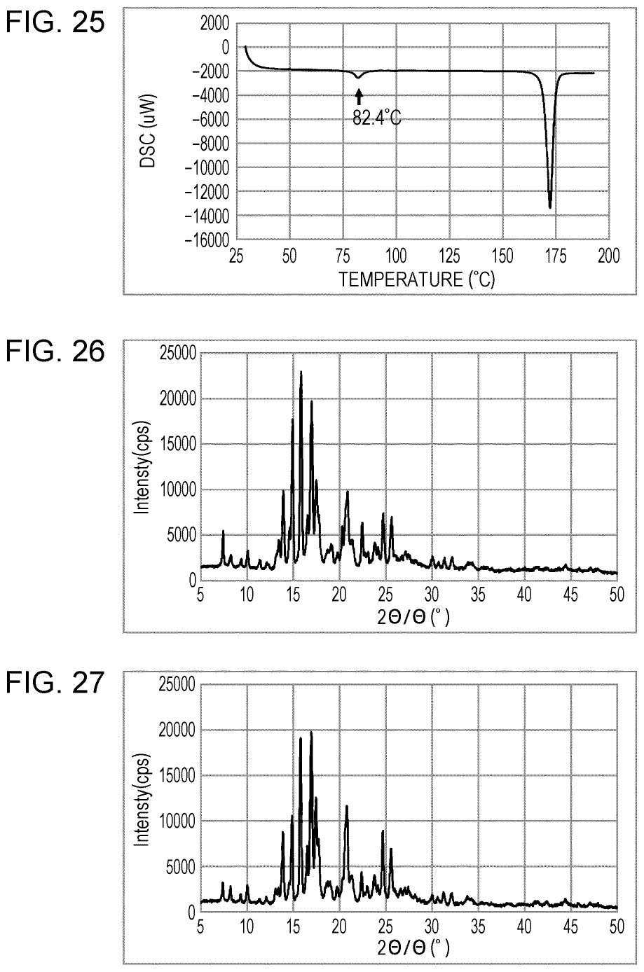

For the crystals obtained in the above drying operation, differential scanning calorimetry (DSC) data of the crystals, [crystal 12-1], [crystal 12-3], and [crystal 12-4], are shown in FIGS. 23, 24, and 25. As the drying proceeded, the amount of the attached solvent on the crystal of compound A obtained in Example 11 decreased, and the size of an endothermic peak attributed to evaporation of the attached solvent and desolvation became smaller in the DSC data, in each of which an endothermic peak attributable to desolvation was observed at 81° C. to 85° C. It was found that the area of an endothermic peak attributed to desolvation (the amount of heat absorption per milligram of crystal) decreased from 11.6 mJ/mg of [crystal 12-3] to 5.5 mJ/mg of [crystal 12-4]. From this, it is believed that the drying operation decreases the content of the solvent solvated with compound A and changes the solvated crystal of compound A to the unsolvated crystal.

It is clear that the crystal of compound A obtained in Example 11 is a crystal I, particularly a crystal I-a.

(Powder X-ray diffraction measurement result)

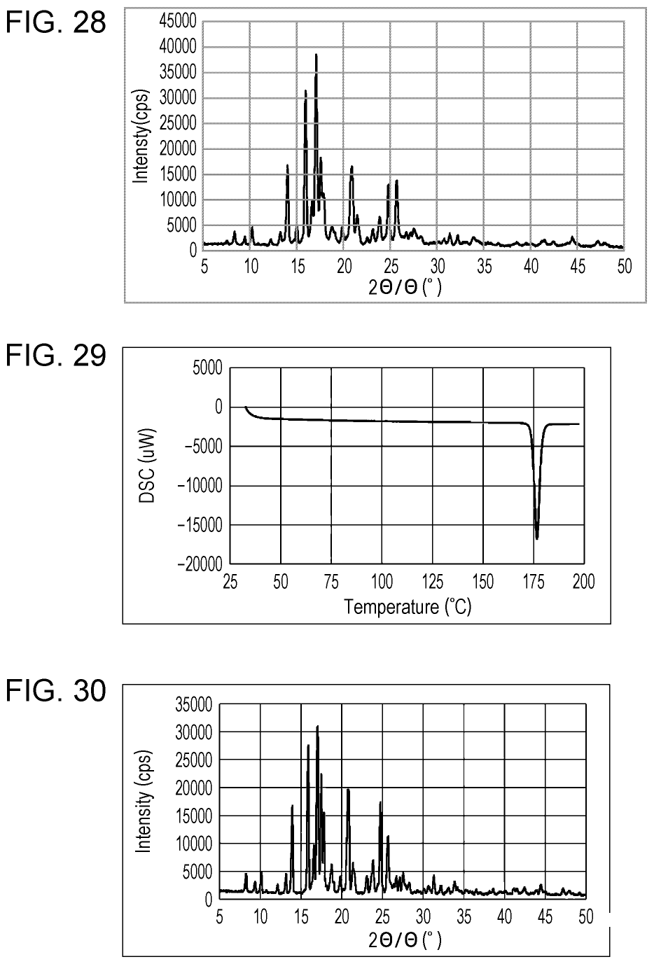

For the crystals obtained in the above drying operation, diffraction angle 2θ (°) of diffraction peaks appearing in powder X-ray diffraction (PXRD) measurement of the crystals, [crystal 12-2], [crystal 12-3], and [crystal 12-4], and peaks whose relative intensity based on a peak with the greatest intensity is 10 or more are shown in Tables 12, 13, and 14, respectively. Their PXRD measurement charts are shown in FIGS. 26, 27, and 28, respectively.

It has become clear that the intensity of peaks at diffraction angles 2θ of 7.5°, 15.0°, and 22.5°, which are peaks appearing in the crystal α′ obtained in Example 11, decreases as the drying proceeds. From this, these peaks are considered to be factors for distinguishing a “crystal α” from a “crystal α′”.

From the result of PXRD analysis of the finally obtained crystal, [crystal 12-4], it is inferred that a trace amount of the crystal α′ has remained because a peak appearing at 15.0°, which is characteristic of the crystal α′ of compound A, is observed in the PXRD chart, but the relative intensity has decreased to less than 10, showing that a crystal α has been produced.

The above results of differential scanning calorimetry analysis and powder X-ray diffraction measurement suggest desolvation due to drying.

TABLE 12

| No. | 2θ (°) | Relative intensity |
|---|---|---|
| 1 | 7.5 | 16 |
| 2 | 13.4 | 22 |
| 3 | 13.9 | 40 |
| 4 | 14.6 | 16 |
| 5 | 14.9 | 68 |
| 6 | 15.9 | 98 |
| 7 | 16.5 | 19 |
| 8 | 17.0 | 100 |
| 9 | 17.5 | 47 |
| 10 | 17.8 | 35 |
| 11 | 19.1 | 16 |
| 12 | 20.3 | 12 |
| 13 | 20.8 | 76 |
| 14 | 21.4 | 28 |
| 15 | 22.4 | 22 |
| 16 | 23.8 | 14 |
| 17 | 24.7 | 26 |
| 18 | 25.6 | 23 |
| 19 | 27.2 | 14 |
| 20 | 34.1 | 15 |

TABLE 13

| No. | 2θ (°) | Relative intensity |
|---|---|---|
| 1 | 13.9 | 35 |
| 2 | 14.9 | 36 |
| 3 | 15.8 | 79 |
| 4 | 16.5 | 20 |
| 5 | 16.9 | 100 |
| 6 | 17.4 | 55 |
| 7 | 17.7 | 35 |
| 8 | 18.6 | 14 |
| 9 | 19.0 | 11 |
| 10 | 20.6 | 28 |
| 11 | 20.8 | 46 |
| 12 | 21.3 | 28 |
| 13 | 22.4 | 15 |
| 14 | 23.8 | 20 |
| 15 | 24.7 | 36 |
| 16 | 25.6 | 28 |
| 17 | 33.9 | 12 |

TABLE 14

| No. | 2θ (°) | Relative intensity |
|---|---|---|
| 1 | 13.9 | 38 |
| 2 | 15.9 | 71 |
| 3 | 16.6 | 20 |
| 4 | 17.0 | 100 |
| 5 | 17.5 | 40 |
| 6 | 17.8 | 30 |
| 7 | 18.8 | 15 |
| 8 | 20.7 | 24 |
| 9 | 20.9 | 33 |
| 10 | 21.4 | 21 |
| 11 | 23.8 | 13 |
| 12 | 24.7 | 27 |
| 13 | 25.6 | 36 |
| 14 | 33.9 | 10 |
| 15 | 44.5 | 13 |

### Example 13: Production of Crystal I·Crystal α

The crystal II (crystal II-a) obtained in Example 1 (33.0 g: solid content 29.8 g) was used as compound A. This crystal and butyl acetate (15.3 g) were added to a 300 mL four-necked flask to prepare a suspension of the crystal. After the flask was purged with nitrogen, the suspension was heated to a liquid temperature of 100° C. to completely dissolve the crystal. The amount of butyl acetate used was a solvent amount 0.5 times the amount of compound A on a weight basis.

Thereafter, to recrystallize compound A from the prepared butyl acetate solution, isooctane (60.2 g) was added over 2 hours and 30 minutes while the solution was stirred with the liquid temperature in the flask being in the range of 92° C. to 101° C. During the addition of isooctane, the solution turned into an oil-out state and then formed a waxy lump once, but the lump was loosened by continuing the stirring. The amount of solvent used for the crystallization operation was 2.5 times the amount of compound A on a weight basis.

Thereafter, the solution was cooled to 74° C. at a rate of 10° C./h. Thereafter, the heater was turned off to allow natural cooling to 25° C. The cooled solution was in a state where a solid was uniformly dispersed. The precipitated solid was separated by filtration with a centrifugal filter, and the flask subjected to filtration and the solid separated by filtration were washed with isooctane (15.5 g). The amount of compound A as a solid adhering to the flask after the filtration operation was quantitatively determined in the above-described manner. As a result, the solid content adhering to the flask was 0.6 g (2.1 wt %) with respect to 29.8 g of the loaded solid content.

The solvent-containing solid (33.8 g) separated by filtration was added to an eggplant-shaped flask and dried for 2.25 hours under a reduced pressure of 0.6 kPa while being heated at 60° C.

Through the above operation, a solid (26.9 g) of compound A, which was the target compound, was obtained. The yield was 90% based on the loaded solid content. The obtained solid contained 0.2 wt % of butyl acetate.

As a result of differential scanning calorimetry analysis, the obtained solid was found to be a crystal having a melting point of 177° C. The differential scanning calorimetry (DSC) data are shown in FIG. 29. From this analytical result, it has become clear that the obtained crystal is a crystal I.

Diffraction angles 2θ (°) of diffraction peaks appearing in powder X-ray diffraction (PXRD) measurement of the obtained crystal and peaks whose relative intensity based on a peak with the greatest intensity is 25 or more are shown in Table 15. The PXRD measurement chart is shown in FIG. 30. From this analytical result, it has become clear that the obtained crystal is a crystal α.

From these analytical results, it has become clear that the obtained crystal is a crystal I (crystal α) of compound A.

TABLE 15

| | | Relative |
|---|---|---|
| No. | 2θ (°) | intensity |
| 1 | 13.9 | 45 |
| 2 | 15.9 | 74 |
| 3 | 17.1 | 100 |
| 4 | 17.5 | 60 |
| 5 | 17.8 | 41 |
| 6 | 20.7 | 64 |
| 7 | 21.4 | 26 |
| 8 | 24.8 | 49 |
| 9 | 25.7 | 34 |

### Example 14: Production of Crystal I·Crystal β

The crystal II (crystal II-a) obtained in Example 1 (33.4 g: solid content 30.1 g) was used as compound A. This crystal and butyl acetate (16.1 g) were added to a 300 mL four-necked flask to prepare a suspension of the crystal. After the flask was purged with nitrogen, the suspension was heated to a liquid temperature of 125° C. to completely dissolve the crystal. The amount of butyl acetate used was a solvent amount 0.5 times the amount of compound A on a weight basis.

To recrystallize compound A from the prepared butyl acetate solution, the solution was cooled to 27° C. over 30 minutes with stirring. Thereafter, isooctane (60.8 g) was added over 2 hours and 30 minutes. During the addition of isooctane, a waxy substance accumulated at the bottom of the flask. Thereafter, stirring was continued for 44 hours, and it was observed that a solid had precipitated and dispersed in the solution. The amount of solvent used for the crystallization operation was 2.6 times the amount of compound A on a weight basis.

After the precipitated solid was poured into a centrifugal filter, the solid in the flask was poured into the centrifugal filter using isooctane (61.3 g) and separated by filtration. The amount of compound A as a solid adhering to the flask after the filtration operation was quantitatively determined in the above-described manner. As a result, the solid content adhering to the flask was 0.9 g (2.5 wt %) with respect to 30.1 g of the loaded solid content.

The solvent-containing solid (51.9 g) separated by filtration was added to an eggplant-shaped flask and dried for 3 hours under a reduced pressure of 1.5 kPa while being heated at 60° C.

Through the above operation, a solid (28.5 g) of compound A, which was the target compound, was obtained. The yield was 93% based on the loaded solid content. The obtained solid contained 1.1 wt % of butyl acetate and 0.1 wt % of isooctane.

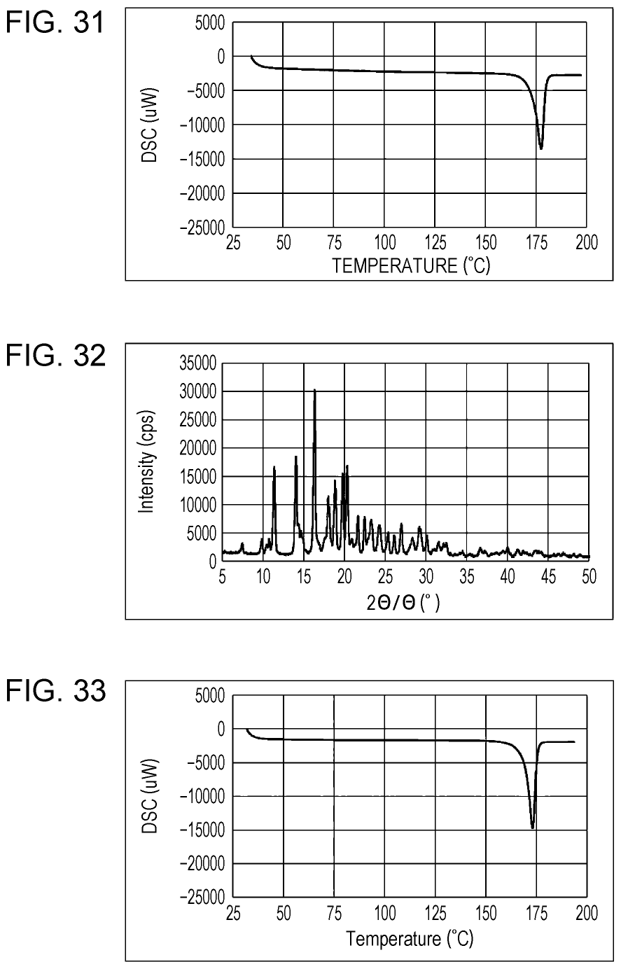

As a result of differential scanning calorimetry analysis, the obtained solid was found to be a crystal having a melting point of 178° C. The differential scanning calorimetry (DSC) data are shown in FIG. 31. From this analytical result, it has become clear that the obtained crystal is a crystal I.

Diffraction angles 2θ (°) of diffraction peaks appearing in powder X-ray diffraction (PXRD) measurement of the obtained crystal and peaks whose relative intensity based on a peak with the greatest intensity is 25 or more are shown in Table 16. The PXRD measurement chart is shown in FIG. 32. From this analytical result, it has become clear that the obtained crystal is a crystal β.

From these analytical results, it has become clear that the obtained crystal is a crystal I (crystal β) of compound A.

TABLE 16

| | | Relative |
|---|---|---|
| No. | 2θ (°) | intensity |
| 1 | 11.4 | 59 |
| 2 | 14.0 | 50 |
| 3 | 14.7 | 31 |
| 4 | 16.3 | 100 |
| 5 | 18.0 | 46 |
| 6 | 18.8 | 48 |
| 7 | 19.8 | 42 |
| 8 | 20.3 | 50 |
| 9 | 23.3 | 27 |
| 10 | 29.2 | 25 |

### Example 15: Production of Crystal I·Crystal β

The crystal α (crystal I) obtained in Example 7 (3.0 g) and tetralin (7.8 g) were added to a 100 mL test tube and dissolved by heating to 125° C. The amount of solvent used was 2.6 times the amount of compound A on a weight basis. After the heating, the solution was cooled at room temperature and stirred overnight.

A precipitated solid was separated with a Kiriyama funnel, and the solid was washed with tetralin (3.1 g) and isooctane (9.2 g). After the crystallization liquid was transferred to the Kiriyama funnel, almost no solid remained in the test tube, and almost no solid adhered to the inner wall of the test tube.

The obtained solid (3.8 g) was added to a 50 mL eggplant-shaped flask and dried for 1 hour under a reduced pressure of 1.0 kPa while being heated at 60° C. Thereafter, the resultant was heated to 100° C. and dried for 3 hours to obtain a solid (2.6 g). The obtained solid contained 2.5% of tetralin.

As a result of differential scanning calorimetry analysis, the obtained solid was shown to be a crystal having a melting point of 173° C. The differential scanning calorimetry (DSC) data are shown in FIG. 33. From this analytical result, it has become clear that the obtained crystal is a crystal I.

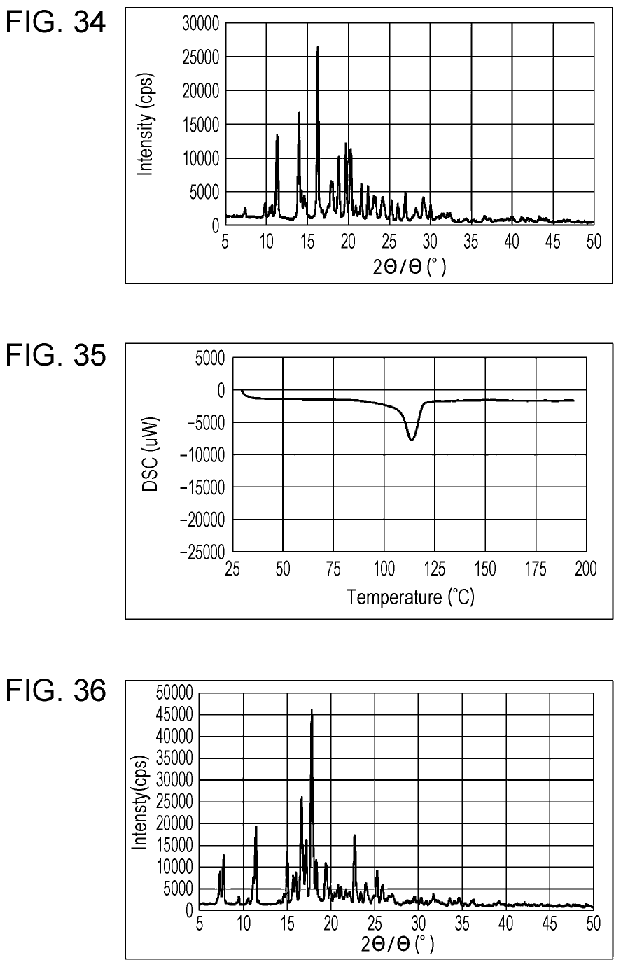

Diffraction angles 2θ (°) of diffraction peaks appearing in powder X-ray diffraction (PXRD) measurement of the obtained crystal and peaks whose relative intensity based on a peak with the greatest intensity is 25 or more are shown in Table 17. The PXRD measurement chart is shown in FIG. 34. From this analytical result, it has become clear that the obtained crystal is a crystal β.

From these analytical results, it has become clear that the obtained crystal is a crystal I (crystal β) of compound A.

TABLE 17

| | | Relative |
|---|---|---|
| No. | 2θ (°) | intensity |
| 1 | 11.3 | 64 |
| 2 | 13.9 | 63 |
| 3 | 14.6 | 26 |
| 4 | 16.3 | 100 |
| 5 | 18.1 | 26 |
| 6 | 18.8 | 39 |
| 7 | 19.7 | 41 |
| 8 | 20.3 | 49 |
| 9 | 23.2 | 29 |

### Example 16: Production of Crystal II·Crystal II-b

The crystal α (crystal I) of compound A obtained in Example 7 (2.1 g) and butyl acetate (2.1 g) were added to a screw tube and dissolved at room temperature. The resulting solution (2.0 g) was portioned into another screw tube and stored in a refrigerator (4° C.) with cyclohexane (5.1 g) added. The amount of solvent used was 6.3 times the amount of compound A on a weight basis.

The precipitated crystal was separated with a Kiriyama funnel. After the crystallization liquid was transferred to the Kiriyama funnel, almost no solid remained in the screw tube, and almost no solid adhered to the inner wall of the screw tube. Thereafter, air-drying was performed on the Kiriyama funnel to obtain a crystal (0.8 g).

Subsequently, the crystal α (crystal I) of compound A obtained in Example 7 (30.0 g) and butyl acetate (35.1 g) were added to a 500 mL four-necked flask and heated to 76° C. After the temperature of the liquid in the flask was decreased to 54° C. by adding cyclohexane (60.2 g), the above crystal (0.03 g) was added, and the heater was turned off to allow natural cooling to 25° C. The amount of solvent used was 3.2 times the amount of compound A on a weight basis.

The precipitated solid was separated by filtration with a centrifugal filter. After the crystallization liquid was transferred to the centrifugal filter, almost no solid remained in the flask, and almost no solid adhered to the inner wall of the flask. The solid separated by filtration (18.5 g) was added to a 300 mL eggplant-shaped flask and dried for 4.3 hours under a reduced pressure of 1.5 kPa while being heated at 65° C.

Through the above operation, a solid (17.2 g) of compound A, which was the target compound, was obtained. The obtained solid contained 12.9 wt % of cyclohexane and 0.8 wt % of butyl acetate.

As a result of differential scanning calorimetry analysis, the obtained solid was shown to be a crystal whose melting point and desolvation temperature were 114° C. The differential scanning calorimetry (DSC) data are shown in FIG. 35. From this result, it has become clear that the obtained crystal is a crystal II.

Diffraction angles 2θ (°) of diffraction peaks appearing in powder X-ray diffraction (PXRD) measurement of the obtained crystal and peaks whose relative intensity based on a peak with the greatest intensity is 25 or more are shown in Table 18. The PXRD measurement chart is shown in FIG. 36. From this analytical result, it has become clear that the obtained crystal is a crystal II-b.

From these analytical results, it has become clear that the obtained crystal is a crystal II (crystal II-b) of compound A.

TABLE 18

| | | Relative |
|---|---|---|
| No. | 2θ (°) | intensity |
| 1 | 11.4 | 43 |
| 2 | 16.6 | 60 |
| 3 | 17.8 | 100 |
| 4 | 22.7 | 27 |

### Example 17: Production of Crystal I·Crystal α by Melting Operation

The crystal II (crystal II-b) obtained in Example 16 (5.1 g) was added to a 50 mL eggplant-shaped flask and dried for 2.5 hours under a reduced pressure of 1.6 kPa while being heated at 108° C. During this process, the solid partially melted into an oil, which was then cooled to obtain a solid (4.3 g). The obtained solid contained 1.3 wt % of cyclohexane and 0.1 wt % of butyl acetate.

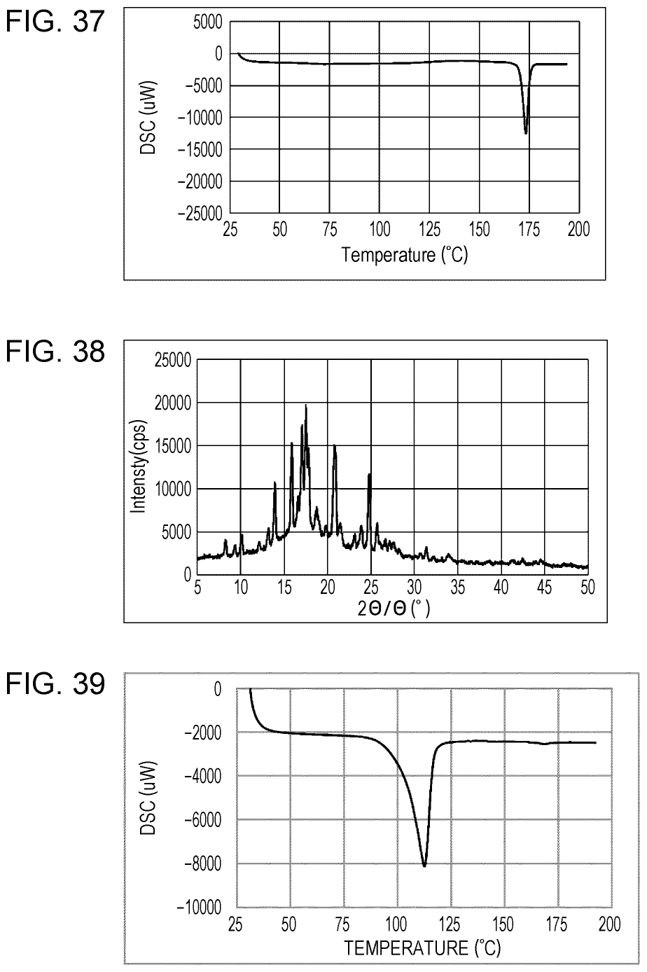

As a result of differential scanning calorimetry analysis, the obtained solid was shown to be a crystal having a melting point of 173° C. The differential scanning calorimetry (DSC) data are shown in FIG. 37. From this analytical result, it has become clear that the obtained crystal is a crystal I.

Diffraction angles 2θ (°) of diffraction peaks appearing in powder X-ray diffraction (PXRD) measurement of the obtained crystal and peaks whose relative intensity based on a peak with the greatest intensity is 25 or more are shown in Table 19. The PXRD measurement chart is shown in FIG. 38. From this analytical result, it has become clear that the obtained crystal is a crystal α (crystal I).

From these analytical results, it has become clear that the obtained crystal is a crystal I (crystal α) of compound A.

TABLE 19

| | | Relative |
|---|---|---|
| No. | 2θ (°) | intensity |
| 1 | 14.0 | 46 |
| 2 | 15.9 | 65 |
| 3 | 17.0 | 92 |
| 4 | 17.5 | 88 |
| 5 | 17.8 | 67 |
| 6 | 18.8 | 26 |
| 7 | 20.8 | 100 |
| 8 | 21.5 | 25 |
| 9 | 24.8 | 58 |
| 10 | 25.7 | 26 |

### Example 18: Production of Crystal II·Crystal II-b

The crystal II (crystal II-a) of compound A obtained in Example 1 (33.3 g: solid content 30.3 g) and butyl acetate (15.5 g) were added to a 300 mL four-necked flask, and the flask was purged with nitrogen by flushing nitrogen to prepare a suspension of the crystal. After the flask was purged with nitrogen, the suspension was heated to a liquid temperature of 100° C. to completely dissolve the crystal. The amount of butyl acetate used was a solvent amount 0.5 times the amount of compound A on a weight basis.

Thereafter, the temperature of the prepared butyl acetate solution was decreased to around 80° C., and to recrystallize compound A, cyclohexane (61.1 g) was added over 2 hours and 30 minutes while the solution was stirred with the temperature of the solution being in the range of 76° C. to 78° C. The amount of solvent used for the crystallization operation was 2.5 times the amount of compound A on a weight basis.

Thereafter, the solution was cooled to 29° C. at a rate of 9° C./h. Thereafter, the heater was turned off to allow natural cooling to 25° C. The cooled solution was in a state where a solid was uniformly dispersed. The precipitated solid was separated by filtration with a centrifugal filter, and the flask subjected to filtration and the solid separated by filtration were washed with cyclohexane (16.4 g). The amount of compound A as a solid adhering to the flask after the filtration operation was quantitatively determined in the above-described manner. As a result, the solid content adhering to the flask was 0.5 g (1.6 wt %) with respect to 30.3 g of the loaded solid content.

The solvent-containing solid (29.5 g) separated by filtration was added to an eggplant-shaped flask and dried for 2.75 hours under a reduced pressure of 0.6 kPa while being heated at 60° C.

Through the above operation, a solid (29.1 g) of compound A, which was the target compound, was obtained. The yield was 82% based on the loaded solid content. The obtained solid contained 13.8 wt % of cyclohexane and 0.7 wt % of butyl acetate.

As a result of differential scanning calorimetry analysis, the obtained solid was found to be a crystal having a melting point of 113° C. The differential scanning calorimetry (DSC) data are shown in FIG. 39. From this analytical result, it has become clear that the obtained crystal is a crystal II.

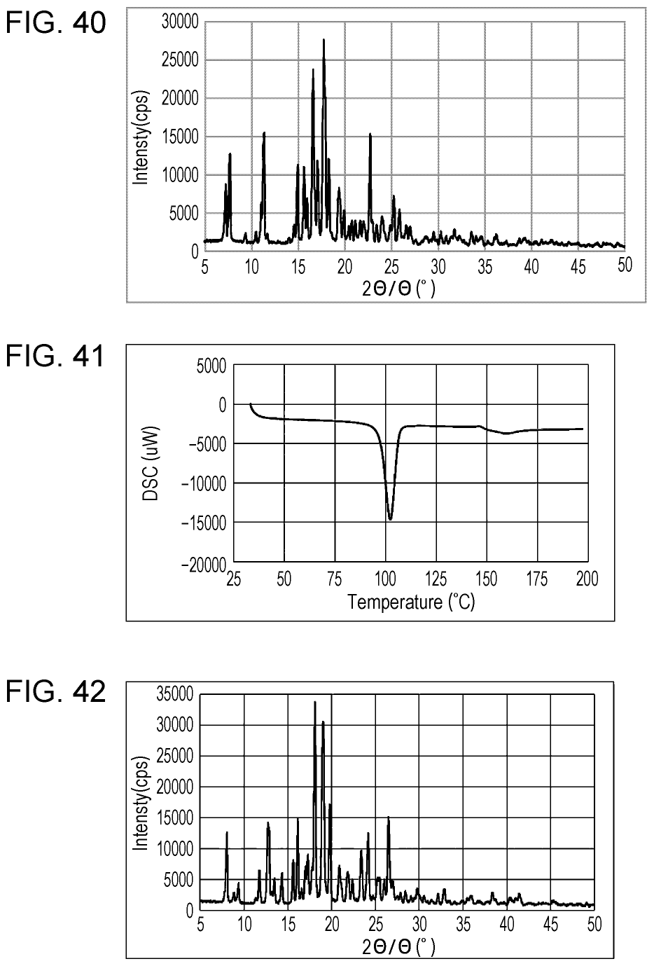

Diffraction angles 2θ (°) of diffraction peaks appearing in powder X-ray diffraction (PXRD) measurement of the obtained crystal and peaks whose relative intensity based on a peak with the greatest intensity is 25 or more are shown in Table 20. The PXRD measurement chart is shown in FIG. 40. From this analytical result, it has become clear that the obtained crystal is a crystal II-b.

From these analytical results, it has become clear that the obtained crystal is a crystal II (crystal II-b) of compound A.

TABLE 20

| | | Relative |
|---|---|---|
| No. | 2θ (°) | intensity |
| 1 | 7.8 | 37 |
| 2 | 11.4 | 48 |
| 3 | 15.0 | 33 |
| 4 | 15.7 | 34 |
| 5 | 16.7 | 81 |
| 6 | 17.2 | 34 |
| 7 | 17.8 | 100 |
| 8 | 18.0 | 39 |
| 9 | 18.3 | 34 |
| 10 | 22.8 | 47 |

### Reference Example 1

The crystal II (crystal II-a) of compound A obtained in Example 1 (0.86 g) and propylene monoglycol methyl ether (PGME) (0.64 g) were added to a screw tube and dissolved at room temperature. The amount of solvent used was 0.8 times the amount of compound A on a weight basis. The resulting solution was left to stand at room temperature for 21 days. The precipitated crystal did not adhere to the screw tube and was uniformly dispersed upon stirring.

The precipitated solid was separated with a Kiriyama funnel and washed with a 50% aqueous PGME solution (4.20 g). After the crystallization liquid was transferred to the Kiriyama funnel, almost no solid remained in the screw tube, and almost no solid adhered to the inner wall of the screw tube. The obtained solid was air-dried on the Kiriyama funnel. As a result of differential scanning calorimetry analysis, the obtained solid (0.29 g) was shown to be a crystal whose melting point and desolvation temperature were 95° C.

Furthermore, the crystal α (crystal I) of compound A obtained in Example 7 (5.01 g) and PGME (5.04 g) were added to a 100 mL test tube and dissolved by heating to 80° C. The liquid temperature was decreased to 27° C., and ultrapure water (3.87 g) and the above crystal (31.3 mg) as a seed crystal were added. The amount of solvent used was 1.8 times the amount of compound A on a weight basis. Thereafter, the heater was turned off to allow natural cooling at 25° C. The precipitated crystal did not adhere to the test tube and was uniformly dispersed upon stirring.

The crystal was separated with a Kiriyama funnel, washed with a 50% aqueous PGME solution (21.76 g), and then air-dried. After the crystallization liquid was transferred to the Kiriyama funnel, almost no solid remained in the test tube, and almost no solid adhered to the inner wall of the test tube. In this manner, a crystal (5.62 g) was obtained.

The obtained crystal (4.77 g) was added to a 50 mL eggplant-shaped flask and dried for 2 hours under a reduced pressure of 1.5 kPa while being heated at 60° C., thereby obtaining a crystal (2.83 g). This crystal contained 15.8% of PGME as a solvent content and 1.6% of water.

### Reference Example 2

The crystal II (crystal II-a) obtained in Example 1 (33.4 g: solid content 30.2 g) was used as compound A. This crystal and propylene monoglycol methyl ether (PGME) (45.4 g) were added to a 300 mL four-necked flask to prepare a suspension of the crystal, and the flask was purged with nitrogen, after which the suspension was heated to a liquid temperature of 98° C. to completely dissolve the crystal. The amount of PGME used was a solvent amount 1.5 times the amount of compound A on a weight basis.

Thereafter, to recrystallize compound A from the prepared PGME solution, water (36.5 g) was added as a poor solvent over 70 minutes. After the addition, the temperature of the solution decreased to 89° C. Thereafter, the solution was cooled to 35° C. at a rate of 10° C./h, and the heater was turned off to allow natural cooling to 25° C. Although stirring was continued for 46 hours from the start of cooling, crystal precipitation was not observed. Thereafter, 0.1 g of the crystal obtained in Reference Example 1 was added as a seed crystal, and the solution was allowed to sit at a temperature of 25° C., as a result of which a crystal precipitated, and the amount of the crystal increased. The precipitated solid was separated by filtration with a centrifugal filter, and the flask subjected to filtration and the solid separated by filtration were washed with water (16.1 g). The amount of compound A as a solid adhering to the flask after the filtration operation was quantitatively determined in the above-described manner. As a result, the solid content adhering to the flask was 0.4 g (1.4 wt %) with respect to 30.1 g of the loaded solid content.

The solvent-containing solid (37.8 g) separated by filtration was added to an eggplant-shaped flask and dried for 3 hours under a reduced pressure of 1.1 kPa while being heated at 60° C.

Through the above operation, a solid (19.3 g) of compound A, which was the target compound, was obtained. The yield was 53% based on the loaded solid content. This crystal contained 16.3% of PGME as a solvent content.

As a result of differential scanning calorimetry analysis, the obtained solid was shown to be a crystal whose melting point and desolvation temperature were 102° C. The differential scanning calorimetry (DSC) data are shown in FIG. 41.

Diffraction angles 2θ (°) of diffraction peaks appearing in powder X-ray diffraction (PXRD) measurement of the obtained crystal and peaks whose relative intensity based on a peak with the greatest intensity is 25 or more are shown in Table 21. The PXRD measurement chart is shown in FIG. 42.

TABLE 21

| | | Relative |
|---|---|---|
| No. | 2θ (°) | intensity |
| 1 | 8.1 | 25 |
| 2 | 12.8 | 45 |
| 3 | 16.2 | 27 |
| 4 | 18.1 | 70 |
| 5 | 19.0 | 100 |
| 6 | 19.8 | 37 |
| 7 | 24.2 | 29 |
| 8 | 26.5 | 34 |

### Reference Example 3

The crystal α (crystal I) obtained in Example 7 (0.67 g) and 1,4-dioxane (1.17 g) were added to a screw tube and dissolved at 35° C. When isooctane (0.40 g) was added to this solution, separation into two layers was observed. This mixed solution (0.22 g) was added to isooctane (2.02 g), and the resulting mixture was left to stand at room temperature for 29 days. A precipitated solid was separated with a Kiriyama funnel, washed with isooctane (5.19 g), and air-dried on the Kiriyama funnel to obtain a solid (0.08 g).

Furthermore, separately, the crystal α (crystal I) obtained in Example 7 (5.11 g) and 1,4-dioxane (3.75 g) were added to a 100 mL test tube and dissolved by heating to 100° C. The amount of 1,4-dioxane used was 0.7 times the amount of compound A on a weight basis, and the amount of solvent used was 2.7 times the amount of compound A on a weight basis. The internal temperature was decreased to 80° C., and isooctane (9.93 g) and the above solid (3.5 mg) as a seed crystal were added. The heater was then turned off to allow natural cooling to 25° C. A precipitated crystal was separated with a Kiriyama funnel, washed with isooctane (28.34 g), and then air-dried to obtain a solid (5.79 g). The obtained solid (4.98 g) was added to a 50 mL eggplant-shaped flask and dried for 1 hour under a reduced pressure of 1.5 kP while being heated at 60° C. to obtain a solid (4.92 g) containing 17.5% of 1,4-dioxane as a solvent content.

### Reference Example 4

The crystal II (crystal II-a) obtained in Example 1 (32.9 g: solid content 29.7 g) was used as compound A. This crystal and 1,4-dioxane (37.6 g) were added to a 300 mL four-necked flask to prepare a suspension of the crystal, and the flask was purged with nitrogen, after which the suspension was heated to a liquid temperature of 99° C. to completely dissolve the crystal. The amount of 1,4-dioxane used was a solvent amount 1.3 times the amount of compound A on a weight basis.

Thereafter, to recrystallize compound A from the prepared 1,4-dioxane solution, isooctane (38.7 g) was added as a poor solvent over 70 minutes. After the addition, the temperature of the solution decreased to 93° C. Thereafter, the solution was cooled to 42° C. at a rate of 10° C./h, and the heater was turned off to allow the solution to cool to 25° C. Although stirring was continued for 24 hours from the start of cooling, crystal precipitation was not observed.

Therefore, 0.1 g of the crystal obtained in Reference Example 3 was added as a seed crystal, and stirring was continued with the temperature of the solution being 25° C., as a result of which a crystal precipitated, and the amount of the crystal increased. To separate the precipitated solid by filtration with a centrifugal filter, the solution was poured into the centrifugal filter, but the solid did not flow out of the flask. Therefore, isooctane (45.7 g) was added into the flask, and the solid was poured out into the centrifugal filter while being loosened. After the solid was separated by filtration, the flask subjected to filtration and the solid separated by filtration were washed with isooctane (73.6 g). The amount of compound A as a solid adhering to the flask after the filtration operation was quantitatively determined in the above-described manner. As a result, the solid content adhering to the flask was 6.5 g (21.9 wt %) with respect to 29.7 g of the loaded solid content.

The solvent-containing solid (56.5 g) separated by filtration was added to an eggplant-shaped flask and dried for 3.3 hours under a reduced pressure of 1.1 kPa while being heated at 60° C.

Through the above operation, a solid (25.9 g) of compound A, which was the target compound, was obtained. The yield was 74% based on the loaded solid content. This crystal contained 15.5% of 1,4-dioxane as a solvent content.

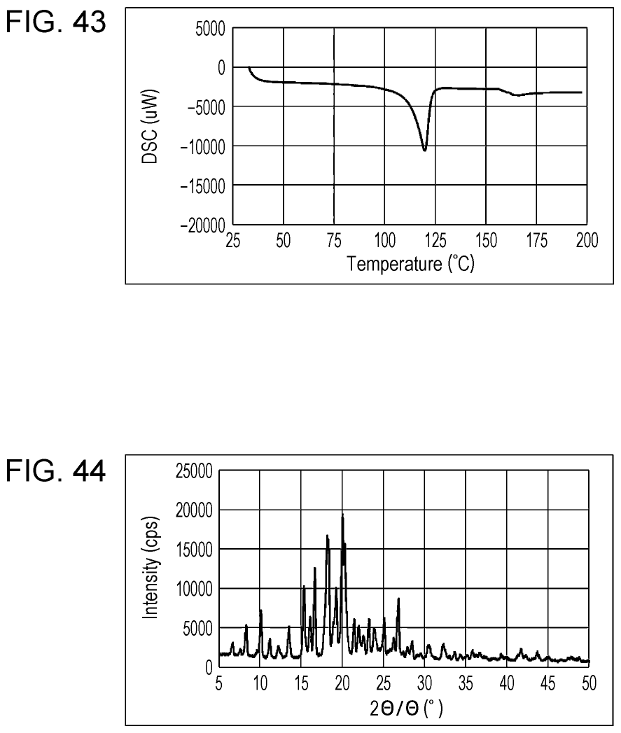

As a result of differential scanning calorimetry analysis, the obtained solid was shown to be a crystal whose melting point and desolvation temperature were 120° C. The differential scanning calorimetry (DSC) data are shown in FIG. 43.

Diffraction angles 2θ (°) of diffraction peaks appearing in powder X-ray diffraction (PXRD) measurement of the obtained crystal and peaks whose relative intensity based on a peak with the greatest intensity is 25 or more are shown in Table 22. The PXRD measurement chart is shown in FIG. 44.

TABLE 22

| | | Relative |
|---|---|---|
| No. | 2θ (°) | intensity |
| 1 | 15.4 | 39 |
| 2 | 16.7 | 43 |
| 3 | 18.1 | 60 |
| 4 | 18.4 | 52 |
| 5 | 19.3 | 50 |
| 6 | 20.0 | 35 |
| 7 | 20.3 | 100 |
| 8 | 26.8 | 27 |

<Bulk density measurement of crystal>

The loose bulk density and the tapped bulk density of the crystals of compound A obtained in Examples 2 to 4, 7, 13, and 18 were measured by the above analysis methods. The results are shown in Table 23.

The crystals obtained in Example 6, Reference Example 1, and Reference Example 2 were in the form of masses at the time of crystal precipitation, filtration, and drying, and needed to be crushed with a spatula for handling. Therefore, the crystals were not in the form of uniform powder, and their measured bulk density values varied depending on the degree of crushing of the masses, so that a proper bulk density measurement could not be performed.

TABLE 23

| | | Loose bulk | Tapped bulk |
|---|---|---|---|
| | | density | density |
| | Type of crystal | (g/cm³) | (g/cm³) |
| Example 2 | I · α | 0.36 | 0.42 |
| Example 3 | I · α | 0.35 | 0.38 |
| Example 4 | I · β | 0.66 | 0.69 |
| Example 7 | mixture of I · α and β | 0.56 | 0.59 |
| Example 13 | I · α | 0.34 | 0.49 |
| Example 18 | II · II-b | 0.38 | 0.45 |

From the results of Examples 1, 16, and 18, it has become clear that the crystal II (crystal II-a, crystal II-b) of compound A according to the present invention has the feature that a crystal of compound A precipitates from a crystallization liquid with a small solvent amount without creating an oil-out state, and the crystal hardly adheres to the inner wall of a flask, is uniformly dispersed in a liquid by stirring, and can be easily separated by filtration. It has become clear that the problems with solids obtained by the compound A crystallization methods known in the art can be solved, and compound A can be produced efficiently in an easy-to-handle form in industrial production.

From the results of Examples 1, 16, and 18, it has become clear that the production methods for obtaining the crystal II (crystal II-a, crystal II-b) of compound A according to the present invention can solve the problems with the crystallization methods known in the art and enable efficient industrial production of compound A as a crystal because compound A can be precipitated as a crystal with a small solvent amount without creating an oil-out state, and the precipitated crystal hardly adheres to the inner wall of a flask, is uniformly dispersed in a liquid by stirring, and can be easily separated by filtration.

Furthermore, from the results of bulk density measurement of the crystal II-b obtained in Example 18, it has become clear that the crystal II-b has a loose bulk density of 0.30 g/cm³ or more, which is a bulk density sufficiently high for industrial production, and it has become clear that the crystal II-b contributes to producing compound A efficiently in an easy-to-handle form.

However, it has become clear that this crystal II (crystal II-a, crystal II-b) has another problem in that it contains much solvent even after being heated and dried under reduced pressure, and its melting point and desolvation temperature are the same, so that the solvent solvated with the crystal cannot be removed without melting.

From the results of Example 8 and Example 17, it has become clear that by melting the crystal II (crystal II-a, crystal II-b) of compound A according to the present invention to remove the solvent and performing cooling, a crystal I·crystal α having a low solvent content can be produced. The crystal I·crystal α thus obtained has a low solvent content, and thus can reduce the amount of exposure to the solvent during the preservation and transportation of compound A and the production of a resin or a derivative using the crystal I·crystal α to contribute to the health of handlers and environmental conservation.

From the results of Examples 9 to 12, it has become clear that the crystal I (crystal α) of compound A according to the present invention has the feature that compound A precipitates as a crystal with a small solvent amount, and the crystal hardly adheres to the inner wall of a flask, is uniformly dispersed in a liquid by stirring, and can be easily separated by filtration. It has become clear that the problems with solids obtained by the compound A crystallization methods known in the art can be solved, and compound A can be produced efficiently in an easy-to-handle form in industrial production.

In addition, it has become clear that the crystal I, particularly the crystal I-a (crystal α′), allows the solvent to be removed without melting even though it is a crystal solvated with methanol. In addition, it has become clear that a crystal I (crystal α) which has a low solvent content and which is in powder form and easy to handle can be produced. That is, it has become clear that the other problems encountered with the crystal II (crystal II-a, crystal II-b) can also be solved.

It has become clear that the methods for obtaining the crystal I-a (crystal α′) of compound A according to the present invention can solve the problems with the crystallization methods known in the art and enable efficient industrial production of compound A as a crystal because compound A can be precipitated as a crystal with a small solvent amount, and the precipitated crystal hardly adheres to the inner wall of a flask, is uniformly dispersed in a liquid, and can be easily separated by filtration.

Furthermore, it has become clear that they are methods that can produce a crystal α′, which allows solvated methanol to be removed without melting even though the precipitating crystal is a crystal solvated with methanol and from which a crystal α, which has a low solvent content and which is in powder form and easy to handle, can be produced.

It has become clear that the crystal α produced by these production methods according to the present invention is a crystal that has a low solvent content and that is in powder form and easy to handle, and enables efficient industrial production of compound A as a crystal.

From the results of Examples 2 to 7 and Examples 13 to 15, it has become clear that the crystal I of compound A according to the present invention has the feature that a crystal of compound A precipitates from a crystallization liquid with a small solvent amount, and the crystal hardly adheres to the inner wall of a flask, is uniformly dispersed in a liquid by stirring, and can be easily separated by filtration. It has become clear that the problems with solids obtained by the compound A crystallization methods known in the art can be solved, and compound A can be produced efficiently in an easy-to-handle form in industrial production.

In addition, it has become clear that compound A can be produced as a crystal that does not solvate with the solvent in a crystallization liquid from the time of precipitation and that has a low solvent content as a result of drying, and that compound A can be produced efficiently with the amount of desolvation-related heat reduced.

Furthermore, from the results of bulk density measurement of the crystals obtained in Examples 2, 3, 4, 7, and 13, it has become clear that the crystals have a loose bulk density of 0.30 g/cm³ or more, which is a bulk density sufficiently high for industrial production, and it has become clear that the crystals contribute to producing compound A efficiently in an easy-to-handle form.

From the results of Examples 2 to 7 and Examples 13 to 15, it has become clear that the production methods for obtaining the crystal I of compound A according to the present invention can solve the problems with the crystallization methods known in the art and enable efficient industrial production of compound A as a crystal because compound A can be precipitated as a crystal with a small solvent amount, and the precipitated crystal hardly adheres to the inner wall of a flask, is uniformly dispersed in a liquid, and can be easily separated by filtration.

Furthermore, it has become clear that they are methods that can produce a crystal α which has a low solvent content and which is in powder form and easy to handle, because a crystal I in powder form that does not solvate with the solvent in a crystallization liquid from the time of precipitation and that allows the solvent to be removed by drying without melting or desolvation can be produced.

Among these production methods, from the results of Examples 2 to 7 and 15, it has also become clear that a crystal of compound A can be precipitated without creating an oil-out state by using ethylbenzene, mixed xylene, or tetralin as a crystallization solvent.

Furthermore, from the results of bulk density measurement of the crystals obtained in Examples 2, 3, 4, 7, and 13, it has become clear that according to these production methods, a crystal of compound A having a loose bulk density of 0.30 g/cm³ or more, which is a high bulk density sufficiently advantageous for industrial production, can be produced, and compound A can be produced efficiently in an easy-to-handle form.

From the results of Examples 2, 3, and 13 and Examples 9 to 12, it has become clear that the crystal α of compound A according to the present invention has the feature that a crystal of compound A precipitates from a crystallization liquid with a small solvent amount, and the crystal hardly adheres to the inner wall of a flask, is uniformly dispersed in a liquid by stirring, and can be easily separated by filtration. It has become clear that the problems with solids obtained by the compound A crystallization methods known in the art can be solved, and compound A can be produced efficiently in an easy-to-handle form in industrial production.

In addition, from the results of Examples 2, 3, and 13, it has become clear that compound A can be produced as a crystal that does not solvate with the solvent in a crystallization liquid from the time of precipitation and that has a low solvent content as a result of drying, and that compound A can be produced efficiently with the amount of desolvation-related heat reduced.

Furthermore, from the results of bulk density measurement of the crystals α obtained in Examples 2, 3, and 13, it has become clear that the crystals α have a loose bulk density of 0.30 g/cm³ or more, which is a high bulk density sufficiently advantageous for industrial production, and it has become clear that the crystals α contribute to producing compound A efficiently in an easy-to-handle form.

From the results of Examples 2, 3, and 13, it has become clear that the production methods for obtaining the crystal α of compound A according to the present invention can solve the problems with the crystallization methods known in the art and enable efficient industrial production of compound A as a crystal because compound A can be precipitated as a crystal with a small solvent amount, and the precipitated crystal hardly adheres to the inner wall of a flask, is uniformly dispersed in a liquid, and can be easily separated by filtration.

Furthermore, from the results of bulk density measurement of the crystals α obtained in Examples 2, 3, and 13, it has become clear that the crystals α have a loose bulk density of 0.30 g/cm³ or more, which is a high bulk density sufficiently advantageous for industrial production, and it has become clear that the crystals α contribute to producing compound A efficiently in an easy-to-handle form.

From the results of Examples 4, 5, 14, and 15, it has become clear that the crystal β of compound A according to the present invention has the feature that a crystal of compound A precipitates from a crystallization liquid with a small solvent amount, and the crystal hardly adheres to the inner wall of a flask, is uniformly dispersed in a liquid by stirring, and can be easily separated by filtration. It has become clear that the problems with solids obtained by the compound A crystallization methods known in the art can be solved, and compound A can be produced efficiently in an easy-to-handle form in industrial production.

In addition, it has become clear that compound A can be produced as a crystal that does not solvate with the solvent in a crystallization liquid from the time of precipitation and that has a low solvent content as a result of drying, and that compound A can be produced efficiently with the amount of desolvation-related heat reduced.

Furthermore, from the results of bulk density measurement of the crystal β obtained in Example 4, it has become clear that the crystal β has a loose bulk density of 0.40 g/cm³ or more, which is a high bulk density more sufficiently advantageous for industrial production, and it has become clear that the crystal β contributes to producing compound A efficiently in an easy-to-handle form.

From the results of Examples 4, 5, 14, and 15, it has become clear that the production methods for obtaining the crystal β of compound A according to the present invention can solve the problems with the crystallization methods known in the art and enable efficient industrial production of compound A as a crystal because compound A can be precipitated as a crystal with a small solvent amount, and the precipitated crystal hardly adheres to the inner wall of a flask, is uniformly dispersed in a liquid, and can be easily separated by filtration.

Furthermore, it has become clear that they are methods that can produce a crystal β which has a low solvent content and which is in powder form and easy to handle, because a crystal β in powder form that does not solvate with the solvent in a crystallization liquid from the time of precipitation and that allows the solvent to be removed by drying without melting or desolvation can be produced.

Furthermore, from the results of bulk density measurement of the crystal β obtained in Example 4, it has become clear that according to this production method, a crystal of compound A having a loose bulk density of 0.40 g/cm³ or more, which is a high bulk density more sufficiently advantageous for industrial production, can be produced, and compound A can be produced efficiently in an easy-to-handle form.

1. A crystal of a trisphenol compound represented by chemical formula (A), having an endothermic peak with a peak top temperature in a range of 168° C. to 180° C., the endothermic peak being determined by differential scanning calorimetry:

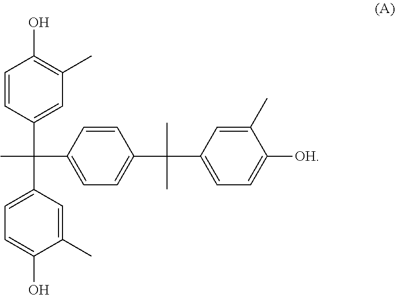 [SMILES: Cc1cc(C(C)(C)c2ccc(C(C)(c3ccc(O)c(C)c3)c3ccc(O)c(C)c3)cc2)ccc1O]

2. (canceled)

3. (canceled)

4. (canceled)

5. A method for producing the crystal according to claim 1, comprising performing crystallization using a solution containing the trisphenol compound represented by chemical formula (A) and an alkylbenzene solvent having a total of 8 to 10 carbon atoms.

6. A method for producing the crystal according to claim 1, comprising performing crystallization by mixing a solution containing the trisphenol compound represented by chemical formula (A) and a carboxylic acid ester solvent having a total of 4 to 8 carbon atoms with a chain aliphatic hydrocarbon solvent having 5 to 10 carbon atoms.

7. A method for producing the crystal according to claim 1, comprising performing crystallization by mixing a solution containing the trisphenol compound represented by chemical formula (A) and an alcohol solvent having 1 to 4 carbon atoms with water.

8. A method for producing the crystal according to claim 1, comprising heating a crystal of the trisphenol compound represented by chemical formula (A) that has one endothermic peak with a peak top temperature in a range of 76° C. to 86° C. and one endothermic peak with a peak top temperature in a range of 168° C. to 180° C., the endothermic peaks being determined by differential scanning calorimetry, to 30° C. or higher and 170° C. or lower.

9. A method for producing the crystal according to claim 1, comprising melting a crystal of the trisphenol compound represented by chemical formula (A) that has peaks at diffraction angles 2θ of 14.8°±0.2°, 15.2°±0.2°, 17.5°±0.2°, and 18.1°±0.2° in a powder X-ray diffraction peak pattern obtained using Cu-Kα radiation to distill off toluene, and performing cooling.

10. A method for producing the crystal according to claim 1, comprising melting a crystal of the trisphenol compound represented by chemical formula (A) that has peaks at diffraction angles 2θ of 11.4°±0.2°, 16.6°±0.2°, and 17.8°±0.2° in a powder X-ray diffraction peak pattern obtained using Cu-Kα radiation to distill off cyclohexane, and performing cooling.

11. A crystal of a trisphenol compound represented by chemical formula (A), having peaks at diffraction angles 2θ of 13.9°±0.2°, 15.8°±0.2°, and 17.0°±0.2° in a powder X-ray diffraction peak pattern obtained using Cu-Kα radiation:

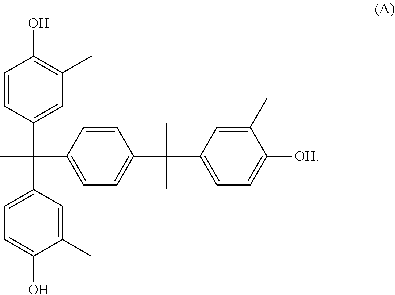 [SMILES: Cc1cc(C(C)(C)c2ccc(C(C)(c3ccc(O)c(C)c3)c3ccc(O)c(C)c3)cc2)ccc1O]

12. (canceled)

13. (canceled)

14. (canceled)

15. A method for producing the crystal according to claim 11, comprising performing crystallization using a solution containing the trisphenol compound represented by chemical formula (A) and an alkylbenzene solvent having a total of 8 to 10 carbon atoms to precipitate the crystal with a temperature of the solution being in a range of 50° C. to 75° C.

16. A method for producing the crystal according to claim 11, comprising performing crystallization by mixing a solution containing the trisphenol compound represented by chemical formula (A) and a carboxylic acid ester solvent having a total of 4 to 8 carbon atoms with a chain aliphatic hydrocarbon solvent having 5 to 10 carbon atoms to precipitate the crystal with a temperature of the solution being in a range of 50° C. to 110° C.

17. A method for producing the crystal according to claim 11, comprising heating a crystal of the trisphenol compound represented by chemical formula (A) that has peaks at diffraction angles 2θ of 13.9°±0.2°, 15.0°±0.2°, 15.8°±0.2°, and 17.0°±0.2° in a powder X-ray diffraction peak pattern obtained using Cu-Kα radiation to 30° C. or higher and 170° C. or lower.

18. A method for producing the crystal according to claim 11, comprising melting a crystal of the trisphenol compound represented by chemical formula (A) that has peaks at diffraction angles 2θ of 14.8°±0.2°, 15.2°±0.2°, 17.5°±0.2°, and 18.1°±0.2° in a powder X-ray diffraction peak pattern obtained using Cu-Kα radiation to distill off toluene, and performing cooling.

19. A method for producing the crystal according to claim 11, comprising melting a crystal of the trisphenol compound represented by chemical formula (A) that has peaks at diffraction angles 2θ of 11.4°±0.2°, 16.6°±0.2°, and 17.8°±0.2° in a powder X-ray diffraction peak pattern obtained using Cu-Kα radiation to distill off cyclohexane, and performing cooling.

20. A crystal of a trisphenol compound represented by chemical formula (A), having peaks at diffraction angles 2θ of 13.9°±0.2°, 15.0°±0.2°, 15.8°±0.2°, and 17.0°±0.2° in a powder X-ray diffraction peak pattern obtained using Cu-Kα radiation:

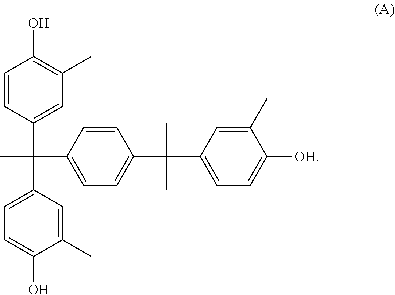 [SMILES: Cc1cc(C(C)(C)c2ccc(C(C)(c3ccc(O)c(C)c3)c3ccc(O)c(C)c3)cc2)ccc1O]

21. A method for producing the crystal according to claim 20, comprising performing crystallization by mixing a solution containing the trisphenol compound represented by chemical formula (A) and methanol with water.

22. A crystal of a trisphenol compound represented by chemical formula (A), having peaks at diffraction angles 2θ of 11.4°±0.2°, 14.0°±0.2°, and 16.3°±0.2° in a powder X-ray diffraction peak pattern obtained using Cu-Kα radiation:

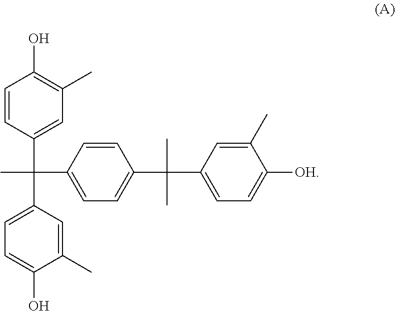 [SMILES: Cc1cc(C(C)(C)c2ccc(C(C)(c3ccc(O)c(C)c3)c3ccc(O)c(C)c3)cc2)ccc1O]

23. (canceled)

24. (canceled)

25. (canceled)

26. A method for producing the crystal according to claim 22, comprising performing crystallization using a solution containing the trisphenol compound represented by chemical formula (A) and an alkylbenzene solvent having a total of 8 to 10 carbon atoms to precipitate the crystal with a temperature of the solution being in a range of 10° C. to 45° C.

27. A method for producing the crystal according to claim 22, comprising performing crystallization by mixing a solution containing the trisphenol compound represented by chemical formula (A) and a carboxylic acid ester solvent having a total of 4 to 8 carbon atoms with a chain aliphatic hydrocarbon solvent having 5 to 10 carbon atoms to precipitate the crystal with a temperature of the solution being in a range of 10° C. to 45° C.

28. A crystal of a trisphenol compound represented by chemical formula (A), having an endothermic peak with a peak top temperature in a range of 103° C. to 116° C., the endothermic peak being determined by differential scanning calorimetry:

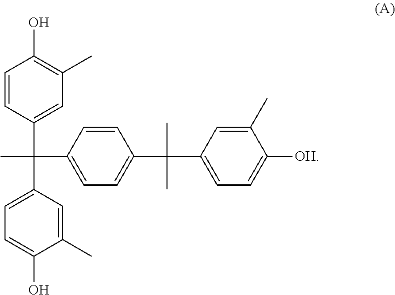 [SMILES: Cc1cc(C(C)(C)c2ccc(C(C)(c3ccc(O)c(C)c3)c3ccc(O)c(C)c3)cc2)ccc1O]

29. The crystal according to claim 28, having peaks at diffraction angles 2θ of 14.8°±0.2°, 15.2°±0.2°, 17.5°±0.2°, and 18.1°±0.2° in a powder X-ray diffraction peak pattern obtained using Cu-Kα radiation.

30. A method for producing the crystal according to claim 28, comprising performing crystallization using a solution containing the trisphenol compound represented by chemical formula (A) and toluene.

31. The crystal according to claim 28, having peaks at diffraction angles 2θ of 11.4°±0.2°, 16.6°±0.2°, and 17.8°±0.2° in a powder X-ray diffraction peak pattern obtained using Cu-Kα radiation.

32. (canceled)

33. A method for producing the crystal according to claim 28, comprising performing crystallization using a solution containing the trisphenol compound represented by chemical formula (A) and cyclohexane.
